(19) United States
(12) Patent Application Publication
(10) Pub. No.: US 2026/0027234 A1
(43) Pub. Date: Jan. 29, 2026

(54) ALZHEIMER'S DISEASE GENE THERAPEUTICS AND METHODS OF USE THEREOF

(71) Applicant: FLORIDA ATLANTIC UNIVERSITY BOARD OF TRUSTEES, Boca Raton, FL (US)

(72) Inventors: Jang-Yen Wu, Boca Raton, FL (US); Howard Prentice, Boca Raton, FL (US); Subash Bhandari, Boca Raton, FL (US)

(21) Appl. No.: 19/279,010

(22) Filed: Jul. 24, 2025

Related U.S. Application Data
(60) Provisional application No. 63/674,976, filed on Jul. 24, 2024.

(51) Int. Cl. A61K 48/00; A61K 9/00; A61K 38/45; A61P 25/28; C12N 15/86

(52) U.S. Cl. CPC A61K 48/005; A61K 9/0043; A61K 38/45; A61K 48/0075; A61P 25/28; C12N 15/86; C12Y 203/01006; C12N 2750/14143

(57) ABSTRACT

The present disclosure provides recombinant vectors encoding a choline acetyltransferase (ChAT) polypeptide, compositions thereof, and methods of use thereof.

### RELATED APPLICATION

This application claims priority to, and the benefit of, U.S. Provisional Patent Application No. 63/674,976, filed Jul. 24, 2024, which is incorporated by reference herein in its entirety.

### REFERENCE TO SEQUENCE LISTING

The sequence listing submitted on Jul. 24, 2025, as an .XML file entitled “11605-068US1_ST26” created on Jul. 8, 2025, and having a file size of 17,046 bytes is hereby incorporated by reference pursuant to 37 C.F.R. § 1.52(e)(5).

### FIELD

The present disclosure provides recombinant vectors encoding a choline acetyltransferase (ChAT) polypeptide, compositions thereof, and methods of use thereof.

### BACKGROUND

Alzheimer's disease (AD) is the most common cause of dementia and it is estimated that 6.2 million Americans age 65 and older are living with AD today. At the molecular and cellular levels, the most striking pathological change in AD is a marked loss of neurons, especially the acetylcholine (Ach) neurons. Despite extensive research, no effective therapeutic intervention is available for AD. The commonly prescribed drugs from AD are either acetylcholine esterase inhibitors e.g., Aricept and Exelon, or NMDA partial antagonist, Namenda. However, their pharmacological effectiveness is quite limited, primarily limited to symptomatic improvement in the early stage of the disease. Thus, there is a need to develop gene therapies focused towards the human choline acetyltransferase (ChAT), the synthesizing enzyme of Ach and a marker of Ach neurons, for the treatment of AD.

The compounds, compositions, and methods disclosed herein address these and other needs.

### SUMMARY

The present disclosure provides methods of treating Alzheimer's disease (AD) using a recombinant adeno-associated virus (AAV) vector expressing a ChAT protein. The present disclosure also provides recombinant AAV vectors and compositions thereof for expressing the ChAT protein.

In some aspects, disclosed herein is a method of treating Alzheimer's disease (AD) in a subject in need thereof, the method comprising administering to the subject a pharmaceutically effective amount of a composition comprising a replication-deficient recombinant adeno-associated virus (AAV) vector and a pharmaceutically acceptable carrier, wherein the AAV vector comprises a polynucleotide encoding a choline acetyltransferase (ChAT) polypeptide.

In some aspects, disclosed herein is a composition comprising a replication-deficient recombinant adeno-associated virus (AAV) vector comprising a polynucleotide and a pharmaceutically acceptable carrier, wherein the polynucleotide encodes a choline acetyltransferase (ChAT) protein.

In some aspects, disclosed herein is a recombinant adeno-associated virus (AAV) vector comprising a polynucleotide encoding a choline acetyltransferase (ChAT) protein.

In some embodiments, the AAV vector comprises at least 70% sequence identity to SEQ ID NO: 1. In some embodiments, the AAV vector comprises at least 80% sequence identity to SEQ ID NO: 1. In some embodiments, the AAV vector comprises at least 90% sequence identity to SEQ ID NO: 1. In some embodiments, the AAV vector comprises SEQ ID NO: 1.

In some embodiments, the AAV vector further comprises a cytomegalovirus (CMV) promoter. In some embodiments, the AAV vector comprises an AAV9 vector.

In some embodiments, the polynucleotide encoding a choline acetyltransferase (ChAT) polypeptide comprises at least 70% sequence identity to SEQ ID NO: 2. In some embodiments, the polynucleotide encoding a choline acetyltransferase (ChAT) polypeptide comprises SEQ ID NO: 2.

In some embodiments, the composition decreases or inhibits AD progression relative to a control. In some embodiments, the method delivers the vector to the subject's central nervous system. In some embodiments, the composition is administered using an ocular gene delivery. In some embodiments, the composition increases expression of the ChAT protein relative to a control composition. In some embodiments, the composition improves mitochondrial dynamics relative to a control composition. In some embodiments, the composition treats or prevents cell death, inflammation, and cognitive dysfunction in the subject relative to a control composition.

In some embodiments, the composition is a ChAT gene therapeutic agent. In some embodiments, the composition is an ocular ChAT gene therapeutic agent. In some embodiments, the pharmaceutically acceptable carrier comprises an excipient, diluent, filler, salt, or buffer.

### BRIEF DESCRIPTION OF FIGURES

The accompanying figures, which are incorporated in and constitute a part of this specification, illustrate several aspects described below.

FIG. 1 shows the acetylcholine synthesis and the role of Choline acetyltransferase (ChAT) in modulating cholinergic pathway.

FIGS. 2A, 2B, 2C, 2D, and 2E show the gene therapy construct as well as transgene and protein expression in the brain of 3×Tg AD mice after the administration of ChAT gene therapy. 3×Tg AD mice were either given AAV2-CMV-GFP (Transgenic control) or AAV-CMV-hChAT (Treatment) as a single dose in the form of 1.5 uL eye drop in the left eye sac on day 0. Mice were sacrificed on day 14. Wild-type (WT) mice received no treatment. FIG. 2A shows a schematic diagram of gene therapy vector constructs used. FIG. 2B shows the 3×Tg mice. FIG. 2C shows the quantification of transgene expression (hChAT) using qRT-PCR. FIG. 2D shows the western blots. FIG. 2E shows the semi-quantitative analysis of the hChAT protein expression. Graphs show densitometry values first standardized to their respective GAPDH band, which were further normalized to WT. Statistical analysis was performed using Ordinary one-way ANOVA with post-hoc multiple comparison analysis using the Tukey-Kramer test. Data are expressed as means±SEMs (**** p≤0.0001, *** p≤0.001, ** p≤0.01, * p≤0.05, ns p≥0.05).

FIGS. 3A, 3B, 3C, and 3D show that ChAT gene therapy reverses early amyloid oligomers and tau phosphorylation in 3×Tg AD mice. FIG. 3A shows the western blot bands, semi-quantitative analysis of these bands showing the relative quantities of amyloid oligomers (FIG. 3B) (A11 antibody) and phosphorylated tau (p-Thr231; FIG. 3C and p-Thr181; FIG. 3D). Graphs shows the densitometry values first standardized to the respective GAPDH band that were further normalized to WT. Statistical analysis was performed using Ordinary one-way ANOVA with post-hoc multiple comparison analysis using the Tukey-Kramer test. Data are expressed as means±SEMs (**** p≤0.0001, ** p≤0.001, ** p≤0.01, * p≤0.05, ns p≥0.05).

FIGS. 4A, 4B, 4C, 4D, 4E, and 4F show that ChAT gene therapy modulates mitochondrial dynamics through regulation of fission, fusion, and mitophagy in young 3×Tg AD mice. FIGS. 4A and 4B show western blots and semi-quantitative analysis of relative ratio of fusion biomarker (Opa1) and fission biomarker (Drp1) in young 3×Tg AD mice. FIGS. 4C and 4D show the semi-quantitative analysis of Opa1 and Drp1. Graphs shows densitometry values first standardized to their respective GAPDH band that were further normalized to WT. Data are represented as the mean±SEM. Statistical analysis was performed using Ordinary one-way ANOVA with post-hoc multiple comparison analysis using the Tukey-Kramer test. Data are expressed as means±SEMs (**** p≤0.0001, ** p≤0.001, ** p≤0.01, * p≤0.05, ns p≥0.05).

FIGS. 5A, 5B, 5C, and 5D show the ChAT gene therapy regulates apoptosis through modulation of Bcl-2 and Bax. FIGS. 5A and 5B show the western blots and semi-quantitative analysis of relative ratio of Bcl-2, a pro survival marker to Bax, an anti-apoptotic marker in young 3×Tg AD mice. FIGS. 5C and 5D show the semi-quantitative analysis of Bcl-2 and Bax. Graphs show densitometry values first standardized to their respective GAPDH band that were further normalized to WT. Data are represented as the mean±SEM. Statistical analysis was performed using Ordinary one-way ANOVA with post-hoc multiple comparison analysis using the Tukey-Kramer test. Data are expressed as means±SEMs (**** p≤0.0001, ** p≤0.001, ** p≤0.01,* p≤0.05, ns p≥0.05).

FIGS. 6A, 6B, 6C, 6D, and 6E show the ChAT gene therapy modulated activity of microglia and astrocytes in young 3×Tg AD mice. FIGS. 6A, 6B, 6C, 6D, and 6E show the western blots, semi-quantitative analysis of densitometry values of ratio of Iab1 and CD68 (FIG. 6B), Iba1 (FIG. 6C), CD68 (FIG. 6D), and GFAP (FIG. 6E). Graphs show densitometry values first standardized to their respective GAPDH band that were further normalized to WT. Data are represented as the mean±SEM. Statistical analysis was performed using Ordinary one-way ANOVA with post-hoc multiple comparison analysis using the Tukey-Kramer test. Data are expressed as means±SEMs (**** p≤0.0001, ** p≤0.001, ** p≤0.01, * p≤0.05, ns p≥0.05).

FIGS. 7A, 7B, 7C, and 7D show the ChAT gene therapy modulates cytokines through TNF-α and Interleukin-10 but not IL-1β in young 3×Tg AD mice. FIG. 7A shows the western blots and semi-quantitative analysis of Tumor Necrosis factor-alpha (TNF-α)(FIG. 7B), Interleukin 1 beta (IL-1β) (FIG. 7C), and Interleukin-10 (IL-10) (FIG. 7D). Graphs show densitometry values first standardized to their respective GAPDH band that were further normalized to WT. Data are represented as the mean±SEM. Statistical analysis was performed using Ordinary one-way ANOVA with post-hoc multiple comparison analysis using the Tukey-Kramer test. Data are expressed as means±SEMs (**** p≤0.0001, ** p≤0.001, ** p≤0.01, * p≤0.05, ns p≥0.05).

FIGS. 8A, 8B, 8C, 8D, and 8E show the Morris water maze test to assess spatial learning and memory in young 3×Tg AD mice. FIG. 8A shows the experimental design of the MWM experiment with location of pre- and post-treatment hidden platform. FIG. 8B shows the mean speed of mice during the Hidden platform training (HPT) and during probe trails. FIGS. 8C, 8D, and 8E show the HPT phase of MWM experiment. Mice on all treatment groups spent a significant percentage of their experiment time in the target quadrant during HPT. When the target from Quadrant 1 (Q1) pre-treatment was relocated to Q3 post-treatment (FIG. 8C), there was a significant decrease in % occupancy of Q1 along with subsequent increase in Q3 occupancy percentage (FIG. 8D). While escape latency decreased as a function of training days both pre- and post-treatment, there was no significance between treatment groups in (FIG. 8E) latency to platform indicating unimpaired spatial reference memory in young 3×Tg AD mice (n=6/group). Statistical analysis was performed using ANOVA with post-hoc multiple comparison analysis using the Tukey-Kramer test. Data are expressed as means±SEMs (**** p≤0.0001, ** p≤0.001, ** p≤0.01, * p≤0.05, ns p≥0.05).

FIGS. 9A, 9B, 9C, 9D, 9E, and 9F show the MWM test to assess working memory and cognitive flexibility in young 3×Tg AD mice. FIGS. 9A and 9B show the working memory in young 3×Tg AD mice by calculating target quadrant occupancy as a percentage of time spent in the target quadrant relative to preceding trail. FIGS. 9C, 9D, 9E, and 9F show that the probe trail was conducted before (FIG. 9C) and after (FIG. 9D) the treatment. Significant differences were observed between WT and 3×Tg AD mice on day 14 post-treatment in terms of target occupancy (FIG. 9E) as well as in cognitive flexibility and memory extinction domain (FIG. 9F) as indicated by the inability of 3×Tg AD mice to remember the new spatial location of the platform along with extinction of older platform location (n=6/group). Statistical analysis was performed using ANOVA with post-hoc multiple comparison analysis using the Tukey-Kramer test. Data are expressed as means SEMs (***** p≤0.0001, ** p≤0.001, ** p≤0.01, * p≤0.05, ns p≥0.05).

FIGS. 10A, 10B, 10C, and 10D show the ChAT gene therapy modulates cell survival in parts through activation of protein kinase B (p-AKT). FIG. 10A shows the western blots and semi-quantitative analysis of ratio of phosphorylated AKT to AKT, p-AKT/AKT (FIG. 10B), p-AKT (FIG. 10C), and AKT (FIG. 10D). Graphs shows densitometry values first standardized to their respective GAPDH band that were further normalized to WT. Data are represented as the mean±SEM. Statistical analysis was performed using Ordinary one-way ANOVA with post-hoc multiple comparison analysis using the Tukey-Kramer test. Data are expressed as means±SEMs (**** p≤0.0001, ** p≤0.001, ** p≤0.01, * p≤0.05, ns p≥0.05).

FIG. 11 shows a contemplated mechanism of action of ChAT gene therapy in “Pre-Clinical” 3×Tag AD mice.

FIGS. 12A, 12B, 12C, 12D, 12E, 12F, and 12G show the APP processing resulting in toxic and non-toxic fragments, observed in 4 months old 3×Tg AD mice. Western blots analysis of brain tissues extracts from ChAT gene therapy treated; transgenic control vector treated as well as WT mice. Two different amyloid antibodies were used to investigate amyloid fragments. A11 oligo antibody from Thermo fisher (#AHB0052) that recognize only Aβ₄₂ (FIG. 12A) and Amyloid antibody from Santa Cruz (#SC28365) (FIG. 12F). A11 antibody showed four different clusters of amyloid beta monomers. No significant difference was seen in C99 APP fragment (FIG. 12B) any difference seen in C83 fragment between treatment groups (FIG. 12C). However, a difference was observed in average optical density between treatment groups on ˜45 kDa band (FIG. 12D) that likely corresponded to cluster of 10×Aβ₄₂ monomers (oligomers) as well as ˜32-36 kDa (FIG. 12E) that most likely corresponded to cluster of 8-9 AB₄₀ monomers or an unknown APP fragment. When examining amyloid antibody from Santa Cruz, however, an ˜75 kDa band was observed that most likely corresponded to C83 fragment of APP, and there was no difference in OD between groups (FIG. 12F and FIG. 12G). Data are represented as the mean±SEM. Statistical analysis was performed using Ordinary one-way ANOVA with post hoc multiple comparison analysis using the Tukey-Kramer test. Data are expressed as means±SEMs (**** p≤0.0001, ** p≤0.001, ** p≤0.01, * p≤0.05, ns p≥0.05).

FIGS. 13A, 13B, 13C, and 13D show the modulation of Endoplasmic Reticulum (ER) stress and Autophagy in the brain of 3×Tg AD after hChAT gene therapy. FIG. 13A shows the western blots and semi-quantitative analysis of markers of ER stress, namely, GRP78 and CHOP, as well as autophagy initiator protein, Beclin-1 and their relative quantity in early AD disease stage. FIGS. 13B and 13C shows the semi-quantitative analysis shows no significant difference between treatment groups in the marker of autophagy (F(2,6)=1.288, p=0.3424) Beclin-1 (FIG. 13B), as well as ER stress GRP78 (FIG. 13C) (F(2,6)=2.554, p=0.1576) and CHOP (F(2,6)=2.143, p=0.1985) (FIG. 13D). Graphs show densitometry values first standardized to their respective GAPDH band that were further normalized to WT. Data are represented as the mean±SEM. Statistical analysis was performed using Ordinary one-way ANOVA with post hoc multiple comparison analysis using the Tukey-Kramer test. Data are expressed as means±SEMs (**** p≤0.0001, ** p≤0.001, ** p≤0.01, * p≤0.05, ns p≥0.05).

FIG. 14 shows the vector map of AAV-CMV-hChAT.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, 100% or more increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The terms “treat,” “treating,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the disclosure may be applied preventively, prophylactically, palliatively or remedially. Treatments are administered to a subject prior to onset (e.g., before obvious signs of AD), during early onset (e.g., upon initial signs and symptoms of AD), or after an established development of AD.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Reference also is made herein to peptides, polypeptides, proteins, and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine, or histidine).

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods consider conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polypeptide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polypeptide.

Variants comprising a fragment of a reference amino acid sequence or nucleotide sequence are contemplated herein. A “fragment” is a portion of an amino acid sequence or a nucleotide sequence which is identical in sequence to but shorter in length than the reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous nucleotides or contiguous amino acid residues of a reference polynucleotide or reference polypeptide, respectively. Fragments may be preferentially selected from certain regions of a molecule, for example the N-terminal region and/or the C-terminal region of a polypeptide or the 5′-terminal region and/or the 3′ terminal region of a polynucleotide. The term “at least a fragment” encompasses the full length polynucleotide or full length polypeptide.

As used herein, a “viral vector” refers to a virus-like particle containing genetic material which can be introduced into a eukaryotic cell without causing substantial pathogenic effects to the eukaryotic cell. A wide range of viruses or viral vectors can be used for transduction but should be compatible with the cell type the virus or viral vector are transduced into (e.g., low toxicity, capability to enter cells). Suitable viruses and viral vectors include adenovirus, lentivirus, retrovirus, among others.

The term “administer,” “administering”, or derivatives thereof refer to delivering a composition, substance, inhibitor, or medication to a subject or object by one or more the following routes: ocular, oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

A “pharmaceutically effective amount” of a drug necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

An “adeno-associated virus” or an “AAV” as used herein refers to a small virus belonging to the genus Dependoparvovirus which are replication-deficient, non-enveloped viruses with linear single-stranded DNA. These viruses are commonly used for creating viral vectors for gene therapy, wherein said viruses can infect dividing and quiescent cells and persist in an extrachromosomal state without integrating into the host genome. AAVs can be engineered to express desired genes or gene products such as mRNA, shRNA, or miRNAs to overexpress or silence a target gene.

A “nucleotide” is a compound consisting of a nucleoside, which consists of a nitrogenous base and a 5-carbon sugar, linked to a phosphate group forming the basic structural unit of nucleic acids, such as DNA or RNA. The four types of nucleotides are adenine (A), cytosine (C), guanine (G), and thymine (T), each of which are bound together by a phosphodiester bond to form a nucleic acid molecule.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined polynucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

### Vectors and Compositions

The present disclosure provides vectors and compositions thereof for treating AD.

In some aspects, disclosed herein is a composition comprising a replication-deficient recombinant adeno-associated virus (AAV) vector comprising a polynucleotide and a pharmaceutically acceptable carrier, wherein the polynucleotide encodes a choline acetyltransferase (ChAT) protein.

In some aspects, disclosed herein is a recombinant adeno-associated virus (AAV) vector comprising a polynucleotide encoding a choline acetyltransferase (ChAT) protein. In some embodiments, the replication-deficient recombinant AAV vector of any preceding aspect is a component of a composition used to treat AD.

AD is a progressive neurological disorder that gradually impairs memory and cognition and recognized as the most common form of dementia affecting over six million people in the United States, usually in elderly subjects. AD is characterized by the buildup of abnormal protein deposits, also known as plaques and tangles, in the brain, which damages and kills brain cells. Although there are many efforts to treat and/or prevent the emergence and progression of AD, there remains no cure for AD. Therefore, there is a need for a therapeutic composition for treating AD.

In some embodiments, the present disclosure provides recombinant vectors, compositions thereof, and method of use thereof comprising a AAV vector, which is a type of parvovirus. This defective parvovirus is a preferred vector because it can infect many cell types and is nonpathogenic to humans. AAV type vectors can transport about 4 to 5 kb and wild type AAV is known to stably insert into chromosome 19. Vectors which contain this site-specific integration property are preferred. An especially preferred embodiment of this type of vector is the P4.1 C vector produced by Avigen, San Francisco, CA, which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically, the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

In some embodiments, the AAV vector is an AAV1, AAV2, AAV4, AAV5, AAV8, AAV9, AAVretrograde (AAVrg), or a variant thereof. In some embodiments, the AAV vector is an AAV9 vector. In some embodiments, the variant of AAV vector includes, but is not limited to a hybrid vector, which is engineered from two or more different AAV serotypes (including, but not limited to AAV1, AAV2, AAV4, AAV5, AAV8, AAV9, or AAVrg) to influence viral transduction efficiencies and viral tropism. In some embodiments, the AAV vector comprises a hybrid AAV vector derived from 2, 3, 4, 5, 6, 7, 8, 9, 10, or more AAV serotypes.

The present disclosure describes the use of promoters that regulate the production or the level of β-amyloid peptides or phosphorylated tau protein (p-tau). To target therapeutic transgene expression that directly induces therapy for preventing important aspects of AD progression, the promoters described herein are employed for genes activating by β-amyloid or p-tau. In some embodiments, the AAV vector further includes, but is not limited to, a cytomegalovirus (CMV) promoter. Non-limiting examples of promoters included in the AAV are promoters for the Pyk2 gene, the SEMA5 gene, and the TREM2 gene. Regarding β-amyloid induced transgene expression, Salazar et al., is incorporated by reference for the teachings of promoters that induce expression of Pyk2 gene, which is risk factor for AD (Salazar S V, Cox T O, Lee S, Brody A H, Chyung A S, Haas L T, Strittmatter S M. Alzheimer's Disease Risk Factor Pyk2 Mediates Amyloid-β-Induced Synaptic Dysfunction and Loss. J Neurosci. 2019 Jan. 23; 39(4):758-772. doi: 10.1523/JNEUROSCI.1873-18.2018. Epub 2018 Dec. 5). Melo de Farias et al. is also incorporated by reference for the teachings of promoters that induced expression of SEMA5, which is a regulator of synaptic plasticity (Melo de Farias A R, Pelletier A, Iohan L C C, Saha O, Bonnefond A, Amouyel P, Delahaye F, Lambert J C, Costa M R. Amyloid-Beta Peptides Trigger Premature Functional and Gene Expression Alterations in Human-Induced Neurons. Biomedicines. 2023 Sep. 18; 11(9):2564. doi: 10.3390/biomedicines11092564). Regarding p-tau induced transgene expression, Eun et al., is incorporated by reference for the teachings of promoters that induce expression of TREM2, which is a neurodegenerative pathway component and related to tau phosphorylation (Eun J D, Jimenez H, Adrien L, Wolin A, Marambaud P, Davies P, Koppel J L. Anesthesia promotes acute expression of genes related to Alzheimer's disease and latent tau aggregation in transgenic mouse models of tauopathy. Mol Med. 2022 Jul. 20; 28(1):83. doi: 10.1186/s10020-022-00506-4).

In some embodiments, the AAV vector of any preceding aspect further comprises one or more regulatory gene components necessary for gene expression, such as for example expression of ChAT. In some embodiments, the one or more regulatory gene component includes, but is not limited to transcription factors, polymerases, enhancers, insulators, and architectural proteins. In some embodiments, the transcription factors comprise activators, enhancers, suppressors, and/or repressors.

In some embodiments, the composition decreases or inhibits AD progression relative to a control. In some embodiments, the composition decreases or inhibits AD progression by 5%, 10%, 15%, 20%, 25%, 30%, 35%, 40%, 45%, 50%, 55%, 60%, 65%, 70%, 75%, 80%, 85%, 90%, 95%, 99%, 100%, more, or less relative to a control.

In some embodiments, the AAV and/or any composition thereof of any preceding aspect can be used to treat and/or prevent initiation or progression of AD.

### Methods for Treating Alzheimer's Disease (AD)

The present disclosure provides methods of treating AD using a recombinant adeno-associated virus (AAV) vector expressing a ChAT protein. The present disclosure also provides recombinant AAV vectors and compositions thereof for expressing the ChAT protein.

In some aspects, disclosed herein is a method of treating Alzheimer's disease (AD) in a subject in need thereof, the method comprising administering to the subject a pharmaceutically effective amount of a composition comprising a replication-deficient recombinant adeno-associated virus (AAV) vector and a pharmaceutically acceptable carrier, wherein the AAV vector comprises a polynucleotide encoding a choline acetyltransferase (ChAT) polypeptide.

As used herein, “replication-deficient” refers to a vector, including but not limited to viral vectors, that comprise viral components, such as for example a viral genome, but are defective in one or more functions that are essential for synthesizing, replicating, and/or assembling viral components. Thus, the vector of the present disclosure comprises viral components, such as a viral genome, but said vector is not capable of displaying typical viral functions. Further, said vectors are capable of eliciting an immune response once administered to a host or subject. Said vector are also optimal for expressing a variety of heterologous genes in a host or subject.

AAV vectors are replication-deficient by the deletion of one or more genes from the viral genome. In some embodiments, the AAV vector comprises a deletion of one or more genes selected from a capsid gene, a replication gene, or an assembly gene.

In some embodiments, the AAV vector comprises at least 70% sequence identity to SEQ ID NO: 1. In some embodiments, the AAV vector comprises 70%, 75%, 80%, 85%, 90%, 95%, 99%, or 100% sequence identity to SEQ ID NO: 1. In some embodiments, the AAV vector comprises SEQ ID NO: 1.

In some embodiments, the polynucleotide encoding a choline acetyltransferase (ChAT) polypeptide comprises at least 70% sequence identity to SEQ ID NO: 2. In some embodiments, the polynucleotide encoding a choline acetyltransferase (ChAT) polypeptide comprises at least 70%, 75%, 80%, 85%, 90%, 95%, 99%, or 100% sequence identity to SEQ ID NO: 2. In some embodiments, the polynucleotide encoding a choline acetyltransferase (ChAT) polypeptide comprises SEQ ID NO: 2.

In some embodiments, the method decreases or inhibits AD progression relative to a control. In some embodiments, the method decreases or inhibits AD progression by 5%, 10%, 15%, 20%, 25%, 30%, 35%, 40%, 45%, 50%, 55%, 60%, 65%, 70%, 75%, 80%, 85%, 90%, 95%, 95%, 99%, 100%, more, or less relative to a control. In some embodiments, the method delivers the vector to the subject's central nervous system.

The composition of any preceding aspect may be administered by any route. In some embodiments, the method comprises administering the composition via an ocular route. As used herein, “ocular” is the medical term referring to tissues of or related to the eye. “Ocular gene therapy” refers to a therapeutic method of administering a genetic composition, such as for example an expression vector or naked DNA, directly to the eye of a subject. In general, the eye is considered a good candidate for gene therapy because it is a small, compartmentalized organ containing numerous blood vessels and nerve endings. The eye also constitutes an optima site for gene therapy due to its anatomy, ease of access, immune-privileged state (limiting the immune responses and inflammatory reactions against the delivered gene product), and tight blood-ocular blood barriers that limit the systemic exposure of the gene product. Viral vector that are optimal for ocular gene therapies, but are not limited to AAVs, adenovirus (AV), and lentiviruses. In some embodiments, the composition is administered using an ocular gene delivery, including but not limited to eye drop delivery, intravitreal delivery, subretinal delivery, and suprachoroidal delivery.

In some embodiments, the composition of any preceding aspect is administered via a variety of routes, including ocular, oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the composition of any preceding aspect (e.g., its stability in the environment of the gastrointestinal tract), the condition of the subject (e.g., whether the subject is able to tolerate oral administration), etc.

The composition of any preceding aspect may be administered in such amounts, time, and route deemed necessary in order to achieve the desired result. The exact amount of the composition of any preceding aspect will vary from subject to subject, depending on the species, age, and general condition of the subject, the severity of AD, the particular composition of any preceding aspect, its mode of administration, its mode of activity, and the like. The composition of any preceding aspect is preferably formulated in dosage unit form for ease of administration and uniformity of dosage. It will be understood, however, that the total daily usage of the composition of any preceding aspect will be decided by the attending physician within the scope of sound medical judgment. The specific therapeutically effective dose level for any particular subject will depend upon a variety of factors including the disease symptoms being treated and the severity of AD; the activity of the composition of any preceding aspect employed; the specific composition of any preceding aspect employed; the age, body weight, general health, sex and diet of the patient; the time of administration, route of administration, and rate of excretion of the specific composition of any preceding aspect employed; the duration of the treatment; drugs used in combination or coincidental with the specific composition of any preceding aspect employed; and like factors well known in the medical arts.

The exact amount of composition of any preceding aspect required to achieve a therapeutically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

In some embodiments, the composition of any preceding aspect is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times. In some embodiments, the composition of any preceding aspect is administered daily. In some embodiments, the composition of any preceding aspect is administered every day, every 2 days, every 3 days, every 4 days, every 5 days, every 6 days, every 7 days, or more. In some embodiments, the composition of any preceding aspect is administered every week, every 2 weeks, every 3 weeks, every 4 weeks, or more. In some embodiments, the composition of any preceding aspect is administered every month, every 2 months, every 3 months, every 4 months, every 5 months, every 6 months, every 7 months, every 8 months, every 9 months, every 10 months, every 11 months, every 12 months, or more. In some embodiments, the composition of any preceding aspect is administered every year, every 2 years, every 3 years, every 4 years, every 5 years, or more.

In some embodiments, the composition increases expression of the ChAT protein relative to a control composition. In some embodiments, the composition increases expression of ChAT by 5%, 10%, 15%, 20%, 25%, 30%, 35%, 40%, 45%, 50%, 55%, 60%, 65%, 70%, 75%, 80%, 85%, 90%, 95%, 99%, 100%, more, or less relative to a control composition. In some embodiments, the composition improves mitochondrial dynamics, including, but not limited to fusion/fission dynamics, apoptotic mechanisms, oxidative stress regulation, oxidative phosphorylation, mitochondrial gene expression, other metabolic mechanism, and other mitochondrial signaling pathways, relative to a control composition. In some embodiments, the composition improves mitochondrial dynamics by 5%, 10%, 15%, 20%, 25%, 30%, 35%, 40%, 45%, 50%, 55%, 60%, 65%, 70%, 75%, 80%, 80%, 85%, 90%, 95%, 99%, 100%, more, or less relative to a control composition. In some embodiments, the composition treats or prevents cell death, inflammation, and cognitive dysfunction in the subject relative to a control composition.

In some embodiments, the composition is a ChAT gene therapeutic agent. In some embodiments, the composition is an ocular ChAT gene therapeutic agent. In some embodiments, the pharmaceutically acceptable carrier is selected from an excipient, a diluent, a salt, a buffer, a stabilizer, a lipid, an emulsion, a nanoparticle, and a cream. One or more active agents can be administered in the “native” form or, if desired in the form of salts, esters, amides, prodrugs, or a derivative that is pharmacologically suitable. Salts, esters, amides, prodrugs, and other derivatives of the active agents can be prepared using standards procedures known to those skilled in the art of synthetic organic chemistry and described, for example, by March (1992) Advanced Organic Chemistry; Reactions, Mechanisms, and Structure, 4^{th} Ed. N.Y. Wiley-Interscience.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Choline Acetyltransferase Gene Therapy Ameliorates Cellular and Behavioral Dysfunctions in Pre-Symptomatic 3×Tag Alzheimer's Disease Mouse Model

In light of the relatively recent understanding that the cholinergic system may play a broader role than classical neurotransmission, it is plausible that the early loss of acetylcholine (ACh), choline acetyltransferase (ChAT), and cholinergic neurons in Alzheimer's disease has a profound effect on AD progression and pathology. Hence, therapies that can directly address this early loss could alter or halt AD progression. To assess the therapeutic benefit of increased ChAT and ACh in the CNS, a ChAT gene therapy was designed herein using a replication-deficient recombinant AAV9 vector under the control of the cytomegalovirus (CMV) promotor encoding the ChAT gene (AAV-CMV-hChAT). Young 3×Tg AD mice received a single dose of this gene therapy via eye drops. The validation of ocular gene delivery system was assessed via functional and biochemical tests, including RT-qPCR, western blotting and the Morris water maze (MWM) test. Statistical analysis was performed using ANOVA. The delivered gene successfully reached the CNS and expressed the hChAT transgene and transcribed the hChAT protein. Compared with transgenic control vector, 3×Tg AD mice treated with ChAT gene therapy showed significantly reduced protein expression levels of Aβ₄₂ oligomers and phosphorylated tau protein (p-Thr231). ChAT gene therapy modulated mitochondrial dynamics, early apoptotic indicators, activity of microglia and astrocytes, and cytokine levels in part through the phosphorylation of AKT. Furthermore, ChAT gene therapy protected these mice against impairment in the cognitive flexibility and memory extinction domains observed in transgenic mice during the reversal phase of the MWM. No significant impairment was observed in spatial reference and working memory in 3×Tg AD mice, although 3×Tg AD mice demonstrated a trend toward impairment. In addition, 3×Tg AD mice demonstrated anxiety-like behavior in the MWM as measured by elevated average swimming speed and no protection was observed in the treatment group. Four month old 3×Tg AD mice exhibited impairment in AD pathology, mitochondrial dynamics, apoptotic modulators, inflammatory cytokines, and glial cells and cognitive flexibility but not spatial reference memory. ChAT gene therapy modulated these early pathological and functional changes in part through activation of PI3K/AKT pathway.

### BACKGROUND

In the absence of effective treatment measures, as many as 139 million people worldwide could be living with Alzheimer's disease (AD) by the year 2050. This chronic neurodegenerative disease places enormous emotional, physical and financial demands on individuals, families, and communities. Although the emergence of disease-modifying therapies provides promise for the future, further research and treatment options are ultimately needed to effectively treat this disease. Amyloid senile plaques and neurofibrillary tangles (NFTs) are the consistent neuropathological features of both early-onset and late-onset AD. Preclinical and longitudinal studies have unambiguously demonstrated that amyloid beta buildup is a critical early pathophysiological hallmark of AD, even if senile plaques may or may not be the underlying cause of the disease progression. This finding is supported by the relatively recent use of positron emission tomography (PET) scanning in vivo for detecting amyloid and hyperphosphorylated tau proteins. The 2018 NIA-AA commission's ATN framework classifies AD as a sequential pathological process instead of a neuropathological state, with the understanding that amyloid beta (Aβ) accumulation occurs slowly over the years. A contemporary understanding of Alzheimer's disease progression recognizes three stages along a pathological continuum: preclinical, prodromal and dementia. While clinical symptoms can be present, diagnosis in these stages increasingly relies on biomarkers (A+/−, T+/−, N+/−) that indicate underlying Alzheimer's pathology. Certain combinations of these biomarkers such as amyloid+ hyperphosphorylated tau+ and neurodegeneration (A+, T+, N+) or A+T+N− or A+T−N+ have been reported to progress to dementia more rapidly than other combinations.

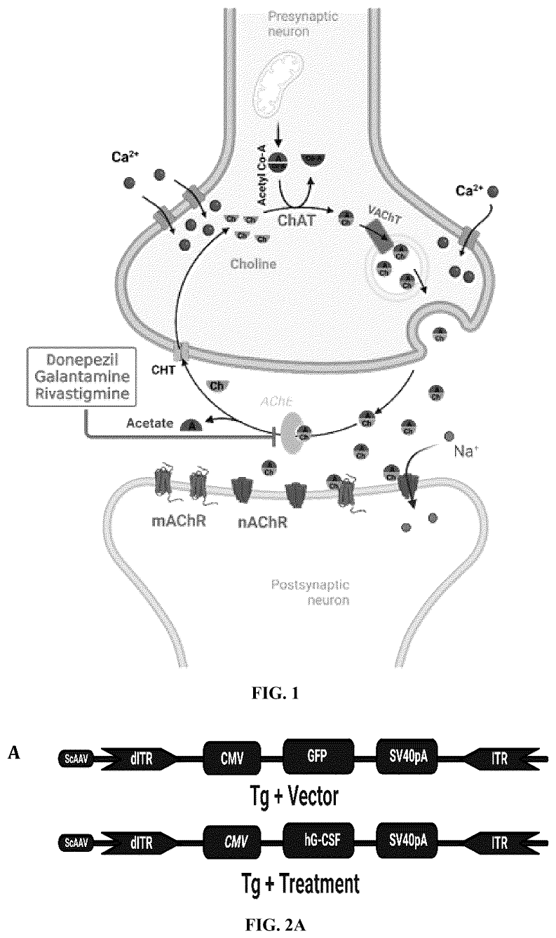

Cholinergic neurotransmission is responsible for modulating important neuronal functions such as learning and memory, attention, stress responses, sleep and wakefulness, sensory information processing, and the parasympathetic “rest and digest” system, including the regulation of heart rate, and other peripheral organs. Cholinergic neurons of the nucleus Basalis of Meynert project to the neocortex and play important roles in higher-order executive functions such as decision making, planning, attention, central executive of working memory, and memory plasticity. In AD patients, these neurons undergo profound degeneration early in the disease process, resulting in cognitive impairments. Although the selective vulnerability of basal forebrain cholinergic neurons was reported as early as the 1970s, the cause of this vulnerability and its overall contribution to AD progression are still poorly understood. Cholinergic neurons release the neurotransmitter acetylcholine (ACh) which is biosynthesized by the enzyme choline acetyltransferase (ChAT) in the presence of acetyl-coenzyme-A and choline (FIG. 1). ChAT is considered an intracellular cytosolic rate limiting enzyme in the biosynthesis of ACh. The physiological roles of ACh, synthesized by ChAT, in mediating skeletal—smooth muscle contractions, neuronal communications in the cholinergic system, and the induction of vasodilation are well established in the literature. Early understanding of the selective degeneration of cholinergic neurons in the basal forebrain led to the development of therapies aimed at preserving or enhancing the physiological amount of ACh in cholinergic synapses. As a result, cholinesterase inhibitors (ChEIs) such as tacrine (1993), donepezil (1996), galantamine (2001), and rivastigmine (2001) were approved by the FDA in the late 1900s and early 2000s, even though tacrine was later recalled (2013) due to safety and efficacy issues. These therapies along with memantine (2003), a partial NMDA receptor antagonist, are being used to manage clinical symptoms in AD patients. These medications, however, provide short term symptomatic relief without altering disease progression. Exploration of cholinergic system and ChEIs treatments studies provided valuable knowledge on the understanding of AD pathology and progression. Importantly, recent longitudinal studies using ChEIs such as galantamine have demonstrated some long-term cognitive benefits partially supporting one of the initial hypotheses that a sustained level of ACh in extracellular synapses could alleviate the cognitive and functional impairment associated with AD patients. These recent findings have led investigators to revisit the cholinergic hypothesis for AD treatment, either independently or in combination with cholinesterase inhibitors.

One biomolecule in the ACh biosynthesis pathway that has received very little attention as a therapeutic agent to enhance the ACh level in AD patients is the enzyme choline acetyltransferase (ChAT). ChAT is the rate-limiting enzyme for ACh synthesis and is primarily believed to be restricted to intracellular cholinergic synaptic terminals. Studies as early as 1977 revealed a significantly reduced level of cholinergic neuronal innervation in the brains of patients with dementia, especially in the neocortex and HPC, and a significantly decreased level of ChAT mRNA expression in the remaining neurons of the basal forebrain regions. Research to address this deficiency has focused primarily on ChEIs, choline, choline transporters (CHT1), and the vesicular acetylcholine transporter (VCHT), but have not targeted ChAT directly, until recently. Some of the early perceived hurdles to considering ChAT as a therapeutic agent for AD were: 1) the understanding that ChAT was restricted to intracellular cholinergic synapses and that it was a membrane-bound enzyme, 2) the limited understanding of the function of ACh and ChAT in the CNS apart from their role in cholinergic neurotransmission, and 3) its inability to cross the BBB. However, some of these assumptions have been revisited recently owing to the overall understanding of the molecular processes underlying disease progression as well as an advancement in the tools and technology. It has been recently reported that in addition to being present in the intracellular space, ChAT is also present in extracellular compartments, including in human plasma and cerebrospinal fluid, and is expressed by acetylcholine-synthesizing T-cell in the spleen, as well as some dendritic cells and macrophages. Vijayaraghavan et al deduced from their findings that extracellular ChAT (eChAT) expression can maintain a steady state level of ACh even in the presence of ACh-degrading enzymes such as AChE and BuChE in the CNS (Vijayaraghavan S, Karami A, Aeinehband S, Behbahani H, Grandien A, Nilsson B, et al. Regulated Extracellular Choline Acetyltransferase Activity—The Plausible Missing Link of the Distant Action of Acetylcholine in the Cholinergic Anti-Inflammatory Pathway. Silman I, editor. PLoS ONE. 2013 Jun. 19; 8(6):e65936). In fact, studies have shown that vasorelaxation in the vasculature is mediated in part through ACh, the level of which is maintained by ChAT, which is present in circulating lymphocytes and blood plasma and that reduced ChAT expression is implicated in hypertension in the periphery. Recent studies have also shed some light on the potential role of ChAT in the immune system, including its impact on blood pressure and the release of cytokines. Cox et al. reported that ChAT-expressing T-cells in the spleen and CD4⁺/ChAT⁺ and CD8⁺/ChAT⁺ expressing T cells are required during chronic infections in an IL-21 dependent manner (Cox M A, Duncan G S, Lin G H Y, Steinberg B E, Yu L X, Brenner D, et al. Choline acetyltransferase-expressing T cells are required to control chronic viral infection. Science. 2019 Feb. 8; 363(6427):639-44). They further established that the increased migration of ChAT expressing T cells was facilitated by brain arterial vasodilation induced by ACh synthesized by ChAT expressing T cells.

The overall understanding of ACh and cholinergic systems in the CNS, until recently, has been limited to cholinergic neurotransmission and synaptic communication, especially communication between the nucleus basalis of Meynert to the neocortex (prefrontal), between the diagonal band of the Broca and medial septum nucleus to the hippocampal regions as well as the amygdala and their involvement in cognition, parasympathetic communication from the brainstem to the PNS and peripheral organs where they are involved in neuromuscular junctions, and the parasympathetic system, and some inhibitory neurotransmission in regions of the brain, such as the striatum. Recent studies have further characterized the additional roles of ACh in modulating non-neuronal cells in the immune response. While approximately 2% of CNS neurons are cholinergic, the majority of non-neuronal cells in the brain are cholinoceptive and sensitive to extracellular changes in ACh. This raises intriguing questions about the role of ACh in the brain beyond classical neurotransmission. Additionally, studies have reported increased plasma ChAT in AD patients while simultaneously showing decreased CSF ChAT, showing a differential synthesis mechanism. In light of recent findings, it is reasonable to contemplate that ACh synthesis and activity may not be limited to cholinergic neurons in the CNS. The first major evidence to support this came from a study in 2011 in which ChAT synthesizing T-cells (ChAT⁺ co-labeled with CD4⁺ and CD8⁺) were isolated from spleens of BALB/c mice (Rosas-Ballina M, Olofsson P S, Ochani M, Valdés-Ferrer S I, Levine Y A, Reardon C, et al. Acetylcholine-Synthesizing T Cells Relay Neural Signals in a Vagus Nerve Circuit. Science. 2011 Oct. 7; 334(6052):98-101), and the ACh released from these T-cells was involved in mediating the innate immune response through the inhibition of TNF-α release from peripheral macrophages (Vijayaraghavan S, Karami A, Aeinehband S, Behbahani H, Grandien A, Nilsson B, et al. Regulated Extracellular Choline Acetyltransferase Activity—The Plausible Missing Link of the Distant Action of Acetylcholine in the Cholinergic Anti-Inflammatory Pathway. Silman I, editor. PLoS ONE. 2013 Jun. 19; 8(6):e65936) and (Hilderman M, Bruchfeld A. The cholinergic anti-inflammatory pathway in chronic kidney disease-review and vagus nerve stimulation clinical pilot study. Nephrol Dial Transplant. 2020 Nov. 1; 35(11):1840-52). These studies addressed two major questions. First, they demonstrated that cells other than cholinergic neurons have the ability to synthesize ChAT and ACh. Second, ChAT is not exclusively located in the cytoplasm of cholinergic neurons within the CNS. This second finding supported earlier reports and has since been confirmed by subsequent studies that have shown the presence of ChAT in B-cells, lymphocytes, astrocytes, microglia, and endothelial cells. Furthermore, a 2013 study by Vijayaraghavan et al (previously cited) at the Karolinska Institute isolated functionally intact and active ChAT protein from the human plasma and CSF of AD patients as well as from healthy controls, demonstrating that ChAT is also present in the extracellular space. Vijayaraghavan et al detected abundant levels of ChAT in the extracellular space that was sufficient to maintain the study state level of extracellular ACh, even in the presence of ChEIs. Subsequent studies have confirmed the presence of ChAT in human plasma and in CSF. These findings support the extracellular presence of ChAT in the CNS as well as in the periphery. Overall, ChAT can be both neurons and nonneuronal cells and it is not exclusively located in the intracellular space of cholinergic neurons.

These findings led to many more questions that need to be clearly addressed. What is the implication of eChAT in the CNS? What is the source of eChAT? Is there a functional difference in the ChAT and ACh synthesized through non-neuronal cells compared to cholinergic neurons? Considering that the majority of non-neuronal cells in the CNS are cholinoceptive, is eChAT or ACh involved in the regulation of cellular responses beyond classical neurotransmission? Is it possible that AD is the disease of neuronal as well as non-neuronal ChAT/ACh deficit? Is a deficit in ChAT/ACh involved in driving the pathological progression of AD? It is believed that all of these are valid questions in light of these findings. Some of these questions are currently being answered by many laboratories around the globe. Recent studies have reported the potential involvement of eChAT in the regulation of immune response, inflammation, vasodilation and blood pressure, among others. For instance, the eChAT is involved in the adaptive immune response to neuroinflammation in the CSF of patients with multiple sclerosis and is strongly corelated with the levels of complement components. Since complement components are expressed by non-neuronal cholinoceptive cells such as microglia and astrocytes in the CNS, it is possible that ChAT influences the activity of glial cells in the modulation of the immune response/inflammation. In fact, herein it is also demonstrated that astrocytes and microglia can synthesize and release ChAT, possibly because glial cells have a complex functional association with ChAT in the modulation of the brain microenvironment. In support of this, subsequent studies have shown that the transplantation of F3.ChAT NSCs in the brains of mice reduced amyloid beta in part through restoring glial functions. Although the inhibition of dentate gyrus granule cells by cholinergic innervations from septo-hippocampal neuronal projections and their role in memory processing are well documented, the role of astrocytes is not well understood. Recent findings by Pabst et al suggested that the inhibitory activity of septo-hippocampal cholinergic neurons occurs not directly through acting on granule cells but rather through α7 nAChR-mediated astrocytic and microglial activation and release of ACh/ChAT.

Although there may or may not be functional differences in ChAT and ACh released by non-neuronal cells vs. neurons, it is important to remember that neurons and glial cells response to different signals; hence, the release of ChAT from neurons vs. non-neuronal cells could have different functional relevance. For example, ChAT released by CD4+ splenocytes is implicated in the regulation of innate immunity, and ChAT released by glial cells in the CNS regulates neuroinflammation. Furthermore, transplantation of neuronal stem cells expressing the ChAT transgene restored brain ACh levels, reduced Aβ levels through the activation of astrocytes and microglia and improved cognitive function. Taken together, these findings strongly support the involvement of ChAT and ACh in the regulation of neuroinflammation in the CNS in addition to their well-established roles in cholinergic neurotransmission, parasympathetic systems and the modulation of neuromuscular junctions and their involvement in the regulation of anxiety, depression, the sleep-wake cycle, memory formation, attention and learning. These findings could reveal the missing link between the degeneration of cholinergic neurons, the downregulation of ChAT and ACh in the CNS and subsequent chronic neuroinflammatory events and brain atrophy in AD patients. These cumulative findings warrant further studies to understand the role of reduced ChAT activity in the brains of AD patients in inflammatory events that are believed to drive the progression of AD pathology. In light of accumulating data showing the extracellular function of ChAT and ACh in modulating non-neuronal systems in the CNS, in addition to the clinical benefits observed in recent longitudinal studies in AD patients taking acetylcholine esterase inhibitors, it is believed herein that the role of ChAT and ACh deficits in AD onset and progression warrants renewed consideration.

The present disclosure assesses the CSF cholinergic index, the ratio of ChAT to acetylcholinesterase (AChE), in the CSF of mild AD patients after the administration of galantamine treatment, there was a positive correlation between galantamine treatment and CSF ChAT levels. Apart from the increase in CSF ChAT activity, there was also improvement in the activity of the Alzheimer's Disease Assessment Scale-Cognitive subscale (ADAS-Cog) and other neuropsychological tests. Furthermore, a subsequent study revealed a decrease in CSF ChAT in AD patients but an increase in CSF ChAT in patients with mild cognitive impairment (MCI) compared to people with subjective cognitive impairment (SCI). The plasma level of ChAT to AChE was the highest in the AD group and the lowest in the SCI group showing a complex relationship between ChAT activity in the CNS and that in the PNS. Aβ deposition in AD patients results in the depletion of ACh and ChAT, followed by an increase in p-tau and cognitive decline. In a study using human neuronal stem cells encoding the ChAT gene (F3.ChAT), the transplantation of F3.ChAT in mice with memory deficits was able to restore learning and memory functions by improving ACh levels, and by restoring microglial functions, such as the downregulation of inflammatory cytokines. In addition to the recovery of learning and memory, F3.ChAT therapy also reduced Aβ and glial fibrillary acidic protein (GFAP) levels, showing that the ChAT has anti-inflammatory and neuroprotective effects. Another study analyzing ChAT expression in the cerebrospinal fluid (CSF) and plasma of AD patients revealed corresponding levels of complement factors, indicating a role for cholinergic signaling in the regulation of inflammation in AD. In light of these recent findings, the present disclosure investigates the effect of ChAT gene therapy in a mouse model of AD.

### Methods

Animals. A Triple-transgenic mice (3×Tg AD) harboring PS1_{M146V}, APP_{swe} and tau_{P301L} transgenes (B6; 129-Tg (APPSwe, tauP301L) 1Lfa Psen1^{tm1MPm}) were used and their corresponding wild-type (WT) controls (C57BL/6×129/Sv, F2 generation, B6129SF2/J) were purchased from Jackson Laboratory and housed under standard laboratory conditions (temperature 23±1° C., a 12-hour day-night cycle, unrestricted access to food and water). The mice were approximately 4 months old at the completion of the experiment. All procedures were conducted according to the guidelines outlined in the institutional Animal Care and Use Committee and approved by the University for the Use and Care of Animals at Florida Atlantic University.

Gene therapy construction. A plasmid containing human ChAT cDNA was purchased from Sino Biological and sent to Vector Bio labs to pack the hChAT gene into a replicant-deficient recombinant AAV9 vector in the presence of the cytomegalovirus (CMV) promotor to produce ChAT gene therapy (AAV-CMV-hChAT). The respective control vector (AAV9-CMV-GFP) was purchased from UNC vector core. 3×TgAD mice (age ˜3.5 months) and their age-matched wild-type controls were randomly assigned to one of two groups (vector control or treatment group) in addition to the WT group. Mice were given a single dose of either AAV9-CMV-hChAT (treatment group) or AAV9-CMV-GFP (vector control) (1×10\hat{ }14 pfu, 1.5 uL) to the left eye sac. The wild-type mice did not receive any treatment. After completion of the Morris water maze (MWM) test, brain tissues were collected for biochemical tests such as western blotting (WB), real-time-quantitative polymerase chain reaction (RT-qPCR), and immunohistochemistry (IHC).

Morris water maze. The Morris water maze (MMW) is a classic rodent test for evaluating spatial reference and working memory in relation to hippocampus-dependent cognitive abilities that are impaired in 3×Tg AD mice. Spatial reference learning is the most standard MWM procedure in which animals learn to use proximal/distal cues around the pool to navigate a direct path to the hidden platform regardless of the drop location. Spatial reversal, often called reversal learning, is an increasingly common and informative protocol used in the MWM test to assess cognitive flexibility and memory extinction in rodents. To investigate spatial reversal, the platform is switched to another quadrant, generally the opposite quadrant, and data from hidden platforms and probe trials were acquired.

The Morris water maze (MWM) procedure was adapted and slightly modified from a published protocol. The experimental tub (120 cm diameter×90 cm height) was filled with water up to 60 cm to ensure that the mice could not jump out of the tub. Four spatial cues (different geometric shapes, same color: white) were hung inside the black curtains in each of the maze quadrants surrounding the tank. The pool was maintained at room temperature (room set at 22° C.) by allowing the water to sit in the tub for at least 1 hour before the start of the experiment. Mice underwent different training phases (acclimation/prehandling, visual platform training (VPT), hidden platform training (HPT), and probe trials) before and after gene therapy administration. A video tracking system (Noldus EthoVision XT, Noldus Information Technology) was used to conduct the experiment. On day −5, the mice were acclimated to the training and holding room and prehandled by the experimenters. Mice were also introduced to the tub water and allowed to swim freely. On day −4 and day −3, each mouse was acclimated to the holding and training room followed by visual platform training. During the visual platform training, the platform (6 cm diameter at the top) was placed at the center of the pool with a water level approximately ½ centimeter below the top of the platform. Mice were placed on the platform and allowed to explore the environment and the platform for 15 seconds, after which they were released into the pool (with the face facing the wall of the tub) and allowed to swim for a maximum of 120 seconds or until they climbed back to the platform. If they did not climb back to the platform at the end of 120 seconds, they were guided to the platform and allowed to stay on the platform for an additional 15 seconds. Starting on day −2, the pool was made opaque using nontoxic white tempera paint (Item #10411697, Michaels) to hide the submerged platform. The platform was placed at the center of quadrant 1 (Q1), 1 cm below the water level. Mice were subjected to 4 trials from different quadrants (semirandom). In each trial of the HPT (day −2 and day −1), the mice were first placed on the platform for 15 seconds, followed by removal and a subsequent drop into the pool from a quadrant drop location (according to the protocol). Each mouse was subjected to 4 HPTs, one from each quadrant on a given day, warmed with warm paper towels and placed in a warmed holding cage (using an electric heating pad). Two days of HPT (8 total trials, four trials/Day, 1 minute inter trial interval) were followed by one probe trial on day 0 before the mice were administered their respective gene therapies. During the probe trial, the platform was first removed from the pool, followed by dropping each mouse in the pool from the opposite quadrant (Q3), and the mice were allowed to explore the pool for 60 seconds. Ten days after the gene therapy administration, the reversal phase of the training was conducted (the platform moved from Q1 to Q3) for five consecutive days. Mice were re-acclimated and trained on the visual platform (center) on days 10 and 11 before HPT started on day 12. From day 12 to day 14, probe trials (1 trial per mouse from the opposite quadrant) were conducted first, followed by HPT (4 trials/mouse, each quadrant, semirandom). The raw data were extracted from the Noldus software and processed using Excel. Differences between the groups were analyzed by repeated-measure ANOVA (if normally distributed, Tukey's multiple comparison test) or the Kruskal-Wallis test (nonparametric, Dunn's multiple comparisons test).

Western Blot. After completion of the behavioral test and at the predetermined timepoints, mice were euthanized for brain tissue collection (western blot, RT-qPCR). Briefly, mice were anesthetized by isoflurane (Phoe-nix), and flash frozen in liquid nitrogen, after which the olfactory bulb and hindbrain tissues were separated and separately stored from the frontal and middle parts of the brain. During tissue processing, brain sections were homogenized in RIPA buffer (Biosciences, Cat #786-490) supplemented with 1% protease and phosphatase inhibitors (Sigma and Thermo Scientific, respectively). The homogenates were centrifuged at 13000 rpm for 30 minutes at 4° C., followed by 30 minutes of shaking in a mini lab roller. The supernatants were collected, and a Bradford assay was performed to determine the protein concentration of the raw sample. Loading samples, containing equal amounts of protein per load were prepared using 2× Laemmli buffer (Bradford, #1610737) supplemented with 5% 2-mercaptoethanol (Sigma, #M3148). For Western blotting (WB) and RT-qPCR, euthanized brains were flash frozen and stored in liquid nitrogen until further processing. Western blots were subjected to SDS-polyacrylamide gel electrophoresis in the laboratory and blotted onto nitrocellulose membranes. mTOR and p-mTOR were detected by WB on a 4% gel and transferred at 4° C. for 24 hours, while lower molecular weight blots were subjected to standard protocol as described above on an 8-20% gel depending on the molecular weight of the antibodies analyzed. Total p-mTOR/mTOR, protein levels were assessed using a Li-COR Revert 700 Kit (Licor, #926-11011, #926-11013, #926-11022). Nitrocellulose membranes were incubated with primary antibodies overnight, followed by 2^{nd} incubations with secondary antibodies for 2 hours at RT. The list of antibodies used and their corresponding concentrations are listed in Table 1. The fluorescence signals were detected using a LI-COR Odyssey Fc system. Densitometric analysis was performed using Image Studio Lite.

Quantitative reverse transcription polymerase chain reaction (RT-qPCR). Total RNA was extracted from the left hemisphere of mouse brains using a RNeasy Mini Kit (Qiagen, CA, USA) according to the manufacturer's instructions. DNA was removed from the samples by using Turbo DNase (Thermo Fisher Scientific). RNA was reverse transcribed utilizing random primers and a thermoscript RT-PCR system (Life Technologies). RT-qPCR was performed as previously described (72,73). The primers used are listed in Table 2. RT-qPCR was performed using an Agilent Aria Mx real-time PCR system.

Statistical Analysis. The data are presented as the mean±SEMs of at least 3 biological replicates. Ordinary one-way ANOVA with Tukey-Kramer post hoc analysis was used to analyze differences between groups. The normality of samples was confirmed using the Shapiro-Wilk test. For the MWM test, two-way ANOVA with repeated measures was used to analyze the training and treatment effects. GraphPad Prism 10.1.2 software was used to report the findings. An Alpha value of 0.05 or less was considered significant for all the statistical tests. **** p≤0.0001, *** p≤0.001, ** p≤0.01, * p≤0.05, ns p≥0.05.

### Results

### Validating Expression of Administered Gene Therapy in Brain Tissue Extracts.

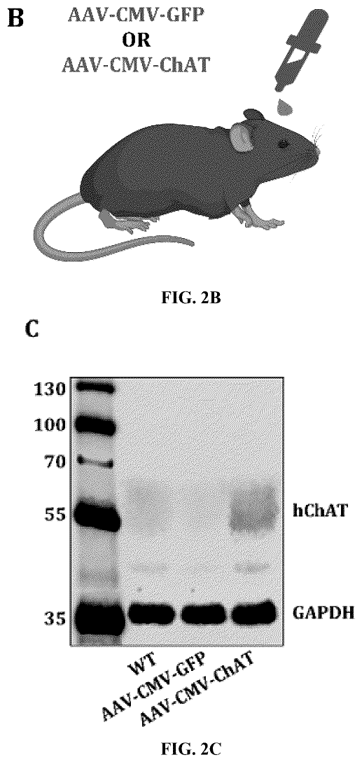

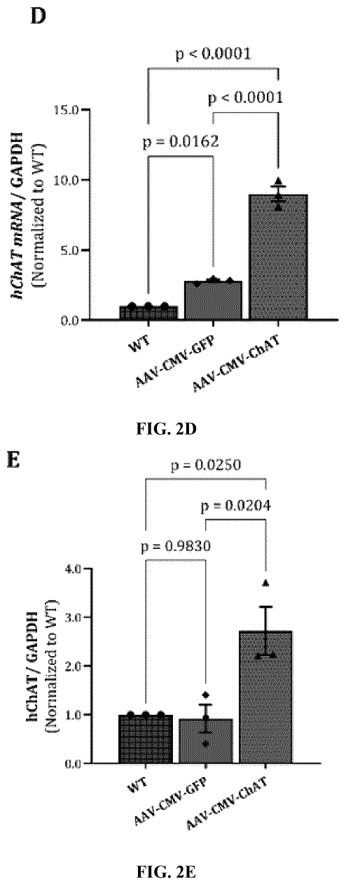

A plasmid containing human ChAT cDNA was purchased from Sino Biological US Inc. and sent to Vector biolabs to further package the hChAT gene into replicant-deficient recombinant AAV9 vector in the presence of a cytomegalovirus (CMV) promotor in order to produce ChAT gene therapy (AAV-CMV-hChAT). The control vector (AAV9-CMV-GFP) was purchased from UNC vector core. The AAV9-CMV-eGFP (Tg+vector) and AAV9-CMV-hChAT (Tg+Treatment) constructs are shown in FIG. 2A. These treatments were administered to 3×Tg AD mice and their corresponding WT on Day 0 as a single dose (3×10\hat{ }9 pfu), and the brain were harvested on day 14 for biochemical analysis after the completion of the behavioral test. Delivery validation and expression of gene therapy delivered through AAV vectors were carried out using the combination of quantitative reverse transcriptase-polymerase chain reaction (RT-qPCR) to quantify mRNA and western blots to quantify the amount of proteins expressed, as demonstrated in FIG. 2. In terms of transgene expression (hChAT mRNA), ordinary one-way ANOVA revealed significant differences between groups (F(2,6)=179.7, p<0.0001) FIG. 2C. Furthermore, the mean transgene expression was significantly greater in the treatment group (8.99±0.53) than in the WT group (1.0, p<0.0001) or the vector control group (AAV-CMV-GFP) (2.78±0.10, p<0.0001). Notably, hChAT mRNA levels in the vector control group were significantly elevated compared to the control group (p=0.0162); however, this difference was not observed at the protein expression level. Protein expression was then measured in these tissues (FIG. 2E) detecting significant differences in protein expression between groups (F(2,6)=9.313, p=0.0145). Further analysis demonstrated that the mean protein expression in the treatment group (2.718±0.50) was significantly greater than in the WT (1, p=0.025) and vector control (0.91±0.29, p=0.0204) groups. There was no significant difference in hChAT protein expression between the WT and vector control (p>0.05). These data demonstrate that gene therapy can be delivered to the brain by noninvasive delivery through the eye sac.

### Elucidating the Role of ChAT Gene Therapy in Modulating Hallmarks of Alzheimer's Disease.

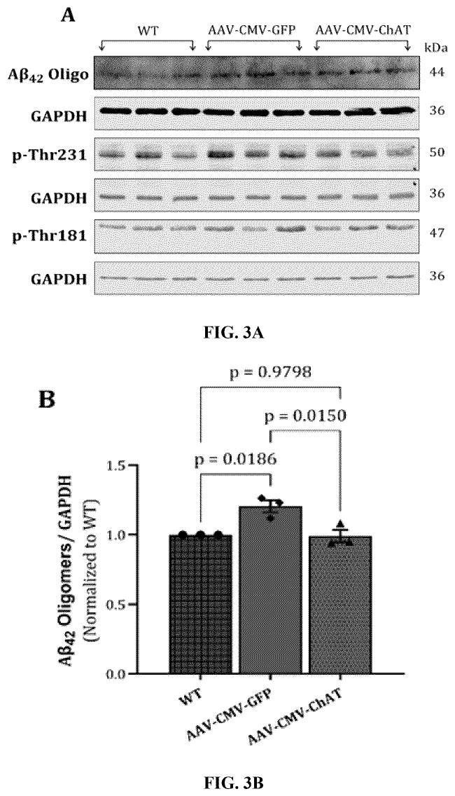

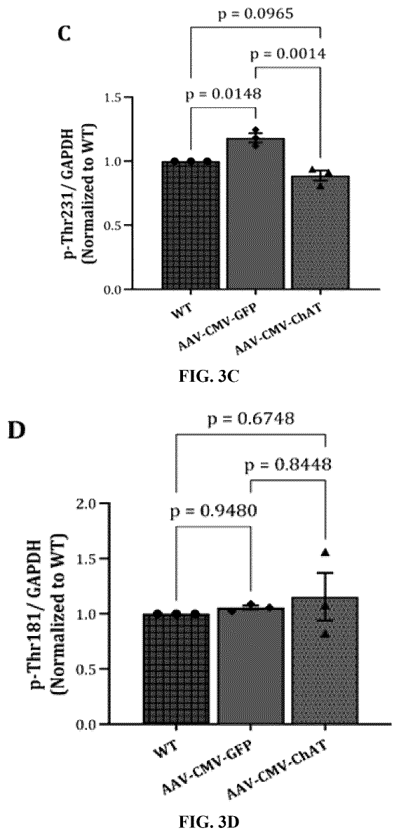

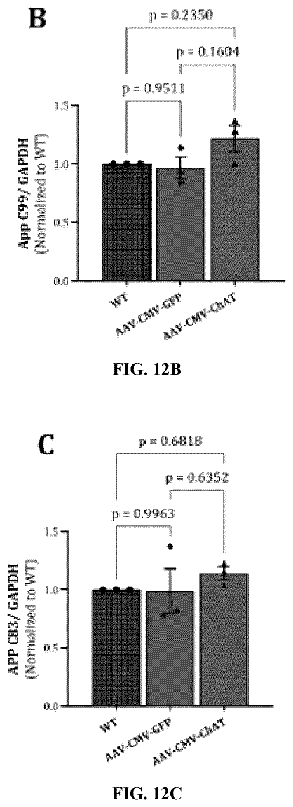

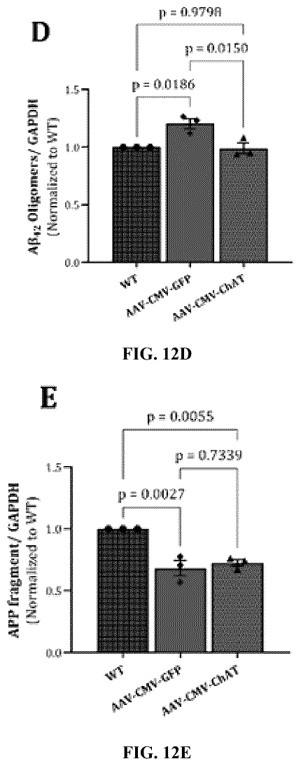

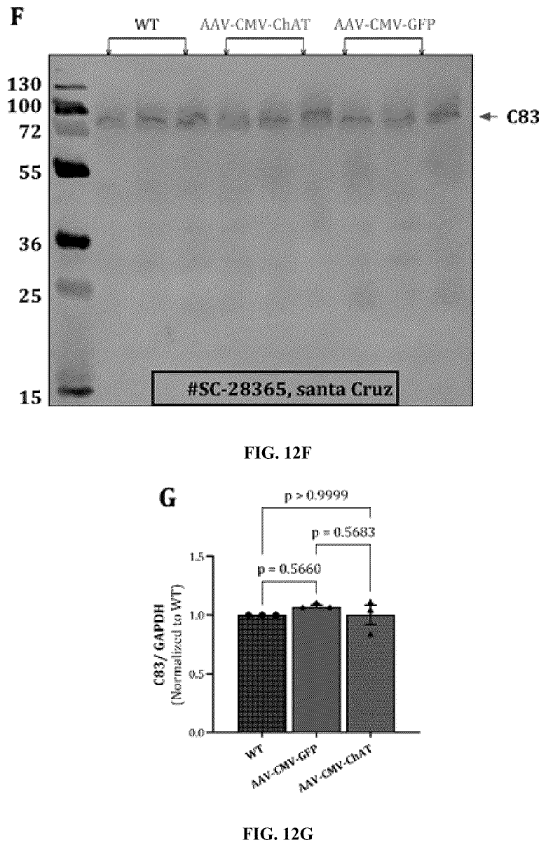

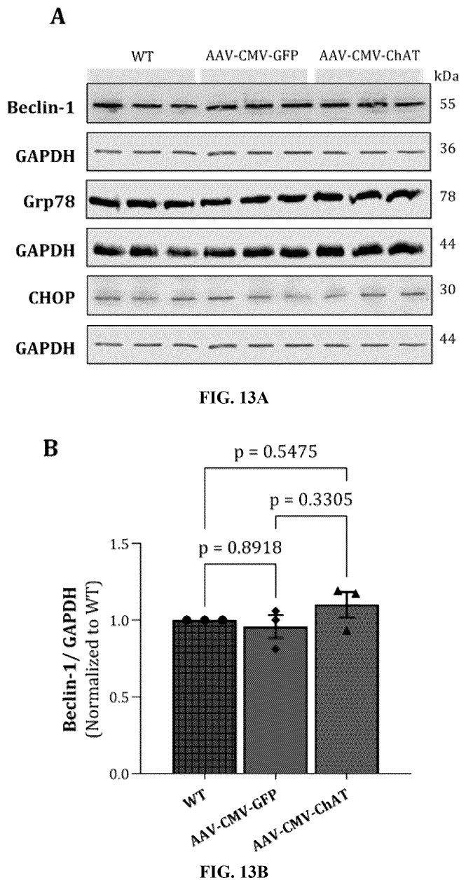

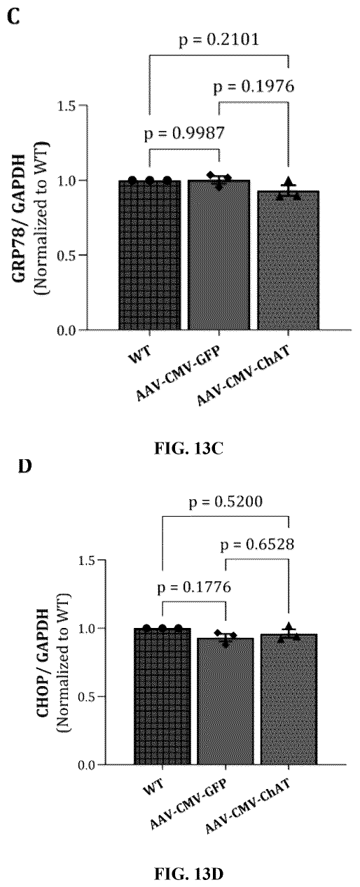

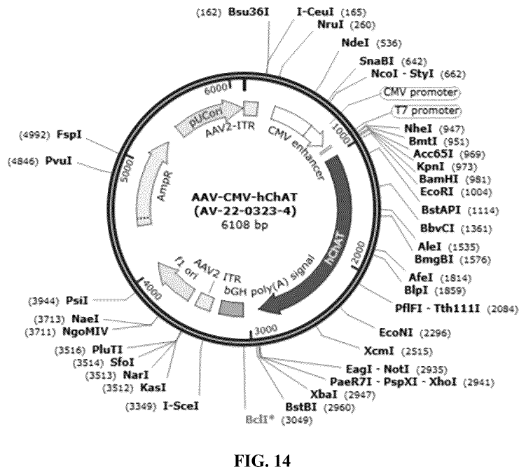

To investigate whether ChAT gene therapy can modulate early signs of Alzheimer's disease, modulation of Aβ oligomers was analyzed using an oligomeric A11 antibody (TF, #AHB0052) along with the modulation of phosphorylated tau products, especially p-Thr231 (Novus Biological, #NB100-82249) and p-Thr181 (Novus Biological, NB100-82245). The A11 antibody recognizes soluble Aβ₄₂ oligomers but not Aβ₄₀, Aβ fibrils or plaques. At least four distinct Western blot bands of A11 (˜100 kDa, ˜68 kDa, ˜45 kDa, and ˜32-36 kDa) were observed (FIG. 12A). The ˜100 kDa band is an APP C99 fragment resulting from the amyloidogenic proteolytic processing of APP by β-secretase, whereas the bands in 68-75 kDa range represent a C83 APP fragment resulting from the nonamyloidogenic proteolytic processing of APP by α-secretase. To confirm that the correct bandwidths were being observed, western blots were performed on brain tissue extracts from age-matched (˜4 months) Tg2576 mice that does not exhibit Aβ oligomers at this age. Bands at 96, 68, and 28-36 kDa range were observed in Tg2576 mice but not in the 45 kDa range, confirming that the 45 kDa band (˜10 mers) represents the Aβ₄₂ oligomers (4.5 kDa/monomers) of interest. Interestingly, western blotting using an amyloid antibody that recognizes amyloid beta and amyloid precursor protein fragments (Santa Cruz, #SC-28365) showed bands corresponding to nonamyloidogenic proteolytic processing of APP by α-secretase (C83 fragment) in the ˜68-75 kDa range but no bands corresponding to APP processing by β-secretase. The Aβ₄₂ oligomers level was significantly greater in ˜4 months old 3×TgAD mice (1.204±0.044) than in WT mice (1, p=0.0186) and that the treatment reduced the level to that of WT mice (0.99±0.046, p=0.0150) (FIG. 3A-3B). Additionally, there was no significant difference between mean OD value of the 68 kDa as well as 96 kDa, however, a significantly reduced level of oligomers was observed in the 34 kDa range (FIG. 12A). Furthermore, the phosphorylation levels of tau protein was measured at different epitopes to determine the presence of paired helical filaments (PHFs) and neurofibrillary lesion formation in 3×Tg AD mice and to determine the effect of ChAT gene therapy. Elevated phosphorylation at Thr231 and Thr181 has a positive correlation to tangle formation and cognitive impairment in AD, with p-Thr231 being an early biomarker of future cognitive impairment, while Thr181 phosphorylation is indicative of some level of tangle and cognitive impairment. Here, 4 month old 3×Tg AD mice had elevated levels of phosphorylation at Thr231 (1.18±0.04) compared those of WT mice (1, p=0.0148) but there was no significant increase in phosphorylation at the Th181 epitope. Importantly, ChAT gene therapy (treatment) significantly reduced the level of phosphorylation at the Thr231 site (0.888±0.040, p=0.0014).

### ChAT Gene Therapy Modulates Mitochondrial Dynamics Through the Regulation of Fission and Fusion and Mitophagy in Young 3×Tag AD Mice.

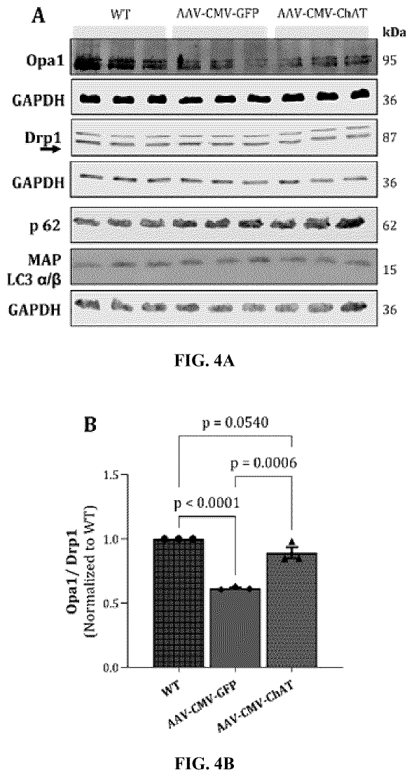

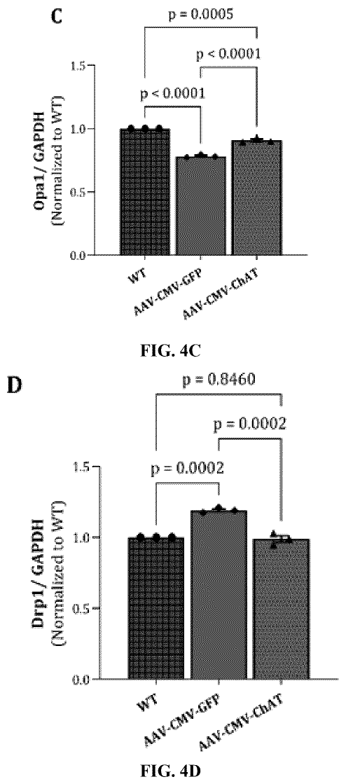

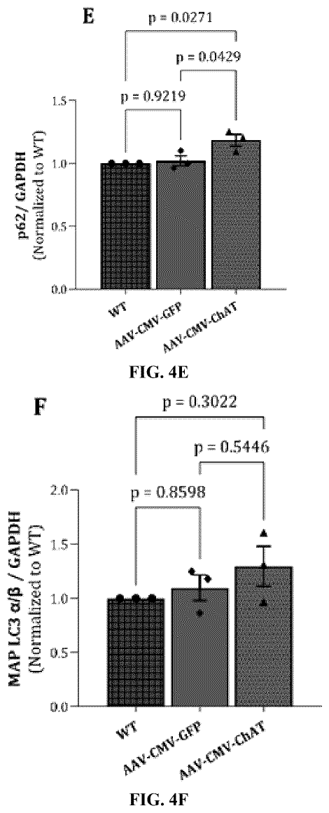

Dysfunctional mitochondrial and oxidative stress predate neuropathological alterations observed in AD and dementia. To analyze the overall mitochondrial state in young 3×Tg AD mice, the expression levels of the mitochondrial-related biomarkers Dynamin-related protein 1 (Drp1) (Abcam, #ab157457) and Optic Atrophy 1 (Opa1) (Abcam, #ab184247), as well as the biomarkers of mitophagy, namely, p62 (Abcam, #ab56416) and MAP LC3 α/β were measured. Mitochondrial fission, the process by which a mitochondrion splits into two parts, is mediated by the cytoplasmic GTPase protein—Drp1, which is recruited by mitochondrial adaptor molecules during fission. When polymerized and activated, Drp1 assembles around fission sites on mitochondria and constricts the mitochondria into two daughter mitochondria. Mitochondrial fusion, the process by which two mitochondria combine to form a single mitochondrion, is mediated in part by Opa1. Here, a significant difference in the mean ratio of Opa1/Drp 1 between the groups (F(2,6)=60.86, p=0.0001), and multiple comparisons further demonstrated that the mean ratio of Opa1 to Dp1 was markedly lower (38% reduction) in the transgenic control mice (0.62±0.01) than in the WT mice (1, p<0.0001) (FIG. 4). This observation of mitochondrial dynamics dysregulation in young 3×Tg AD mice indicates mitochondrial stress and oxidative stress responses exacerbate early pathological changes in AD. ChAT gene therapy was able to rescue mitochondrial fusion away from fission (0.89±0.04, p=0.0006) (FIG. 4A-4B). Furthermore, the individual ratios of fusion and fission biomarkers were measured. There was a 22% reduction in Opa1 in the transgenic control group (0.78±0.01) compared to that in the WT group (1, p<0.0001), while Drp1 was elevated by 19% in the transgenic control group (1.19±0.01) compared that in the WT group (1, p=0.0002) (FIG. 4A, 4C-4D). In both cases, treatment significantly reversed these alterations in Opa1 (0.91±0.012, p<0.0001) and Drp1 (0.989±0.023, p=0.000). Furthermore, biomarker changes associated with mitophagy were analyzed. There was a significant difference in p62 expression between the treatment groups (F(2,6)=7.72, p=0.0219), and further multiple comparisons demonstrated that there was no difference in the mean p62 expression between the WT and transgenic control groups (1.02±0.041, p=0.9219), but ChAT treatment (1.184±0.048) significantly increased the p62 level compared to that in the transgenic control group (p=0.0429) or the WT group (p=0.0271). In terms of LC3, elevated expression in both the transgenic control and treatment groups were detected, but the difference was not significant (F(2,6)=1.396, p=0.3179).

### ChAT Gene Therapy Regulates Apoptosis Through the Modulation of Bcl-2 and Bax in Young 3×Tag AD Mice.

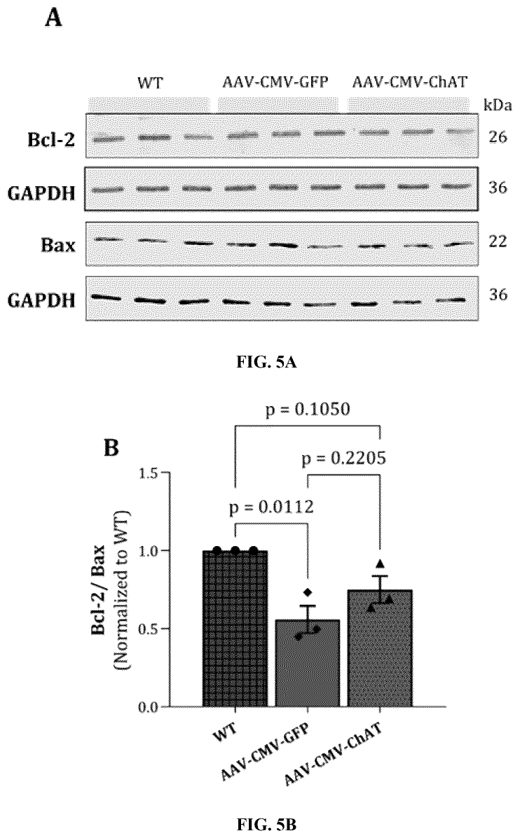

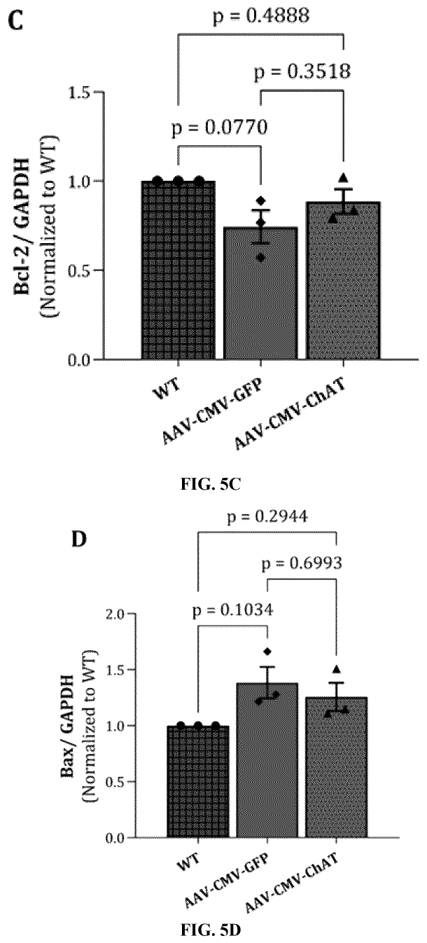

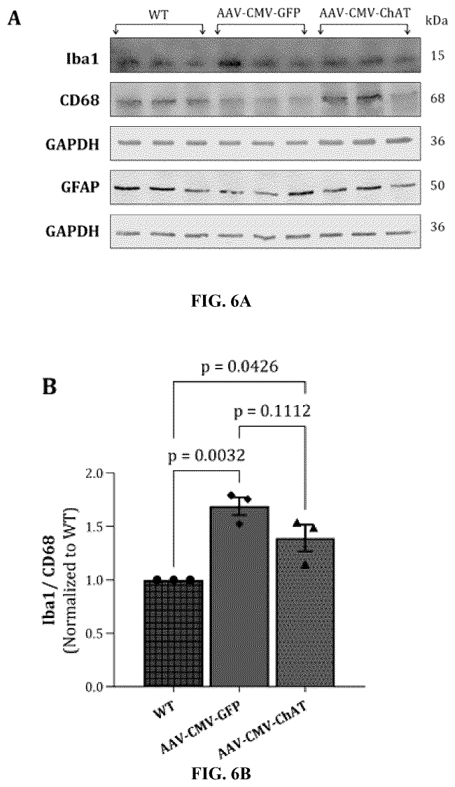

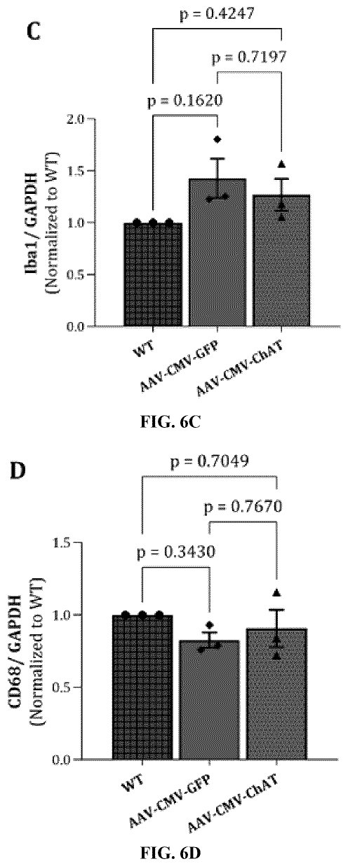

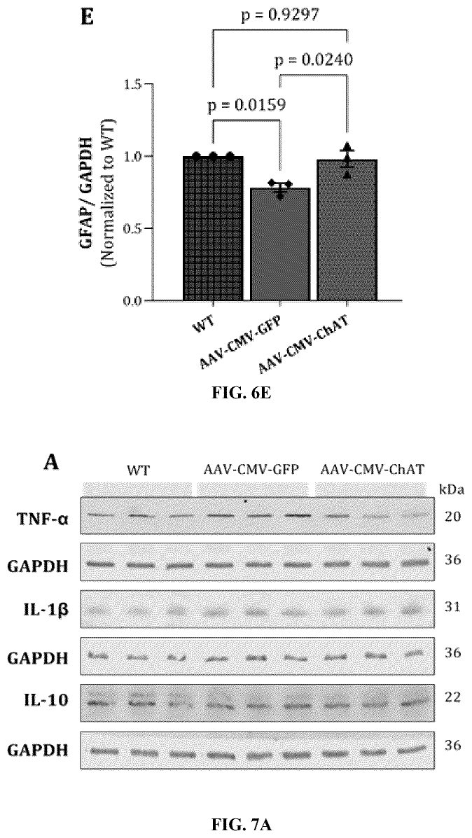

The relative ratio of Bcl-2 to Bax is a standard technique used to assess apoptosis in brain tissues and cellular extracts. The B-cell lymphoma-2 (Bcl-2) family of proteins regulates cell death processes, with Bcl-2 serving as a prosurvival biomarker, whereas Bax promotes cell death. Here, western blot analysis was used to assess apoptotic activity by measuring the relative ratio of Bcl-2 to Bax in young 3×Tg AD mice and to assess the ability of ChAT gene therapy to modulate apoptosis (FIG. 5). Ordinary one-way ANOVA revealed a significant difference in the mean optical densitometry ratio of Bcl-2 to Bax between the groups (F(2,6)=9.613, p=0.0135) (FIG. 5A-5B). By analyzing the differences between groups using the Tukey-Kramer multiple comparison test, there was a significant reduction in the Bcl-2/Bax ratio of as much as 44% in the transgenic control animals (0.56±0.09) compared to that in the WT animals (1, p=0.0112). Treatment seemed to promote toward survival by increasing the Bcl-2/Bax ratio, but the effect was not significant (0.75±0.09, p=0.2205). Furthermore, when analyzing the biomarkers individually, Bcl-2 and Bax were both altered in the transgenic control mice, with Bcl-2 showing slight (26% reduction) but nonsignificant depression compared to that in the WT mice (0.7426±0.09, p=0.077) and Bax showing no significant (38% elevation) increase compared to that in the WT mice (1.384±0.14, p=0.1034) (FIG. 5A, 5C-5D). Treatment reversed the changes in both Bcl-2 (0.89±0.07, p=0.3518) and Bax (1.256±0.13), but the effects was not significant.

### Efficacy of ChAT Gene Therapy in Modulating the Activity of Astrocytes and Microglia.

To assess the activity of immune modulators in these young 3×Tg AD mice, brain tissue samples were probed for the expression on the microglial markers—Ionized calcium binding adaptor molecule 1 (Iba1) and cluster of differentiation 68 (CD68), along with the astrocytic marker, glial fibrillary acidic protein (GFAP). Iba1 is a pan-microglial marker expressed by the majority of microglia at varying intensities. The intensity of Iba1+ microglia has been a subject of latest debate, with some recent convincing evidence demonstrating that the number of migrating microglia decreased in AD patients, as microglia acquires a CD68+ phagocytic phenotype and eventually an amoeboid phenotype. Activated microglia are CD68+ and play a major role in inflammation and AD. Herein, there was a significant difference in the mean ratio of Iba1 to CD68 between groups (ordinary one-way ANOVA, F(2,6)=15.96, p=0.0032). Furthermore, Tukey-Kramer multiple comparison tests revealed that the mean ratio of Iba1/CD68 was significantly greater in transgenic control mice (1.689±0.08) than in WT mice (1, p=0.0032). In mice treated with ChAT gene therapy, there was a decrease in the level of Iba1/CD68 (1.392±0.124), but the effect was not significant (p=0.1112). When looking at these biomarkers individually, however, there were not any significant difference between groups in either Iba1/GAPDH (F(2,6)=2.336, p=0.1777) or the (F(2,6)=1.173, p=0.3715). In terms of astrocytes, there was a significant difference in the mean densitometry values between the groups (F(2,6)=10.02, p=0.0122). Intertreatment group comparisons revealed significant downregulation of GFAP in the transgenic control group (0.782±0.03) compared to the WT group (1, p=0.0159), and ChAT gene therapy significantly increased the level of GFAP (0.9803±0.059, p=0.024).

### ChAT Gene Therapy Modulates Cytokines Through the Regulation of TNF-α and Interleukin-10 but not IL-1β in Young 3×Tag AD Mice.

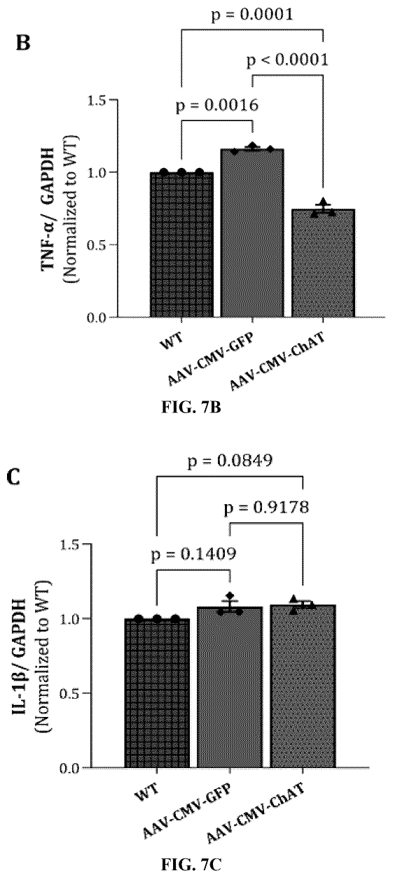

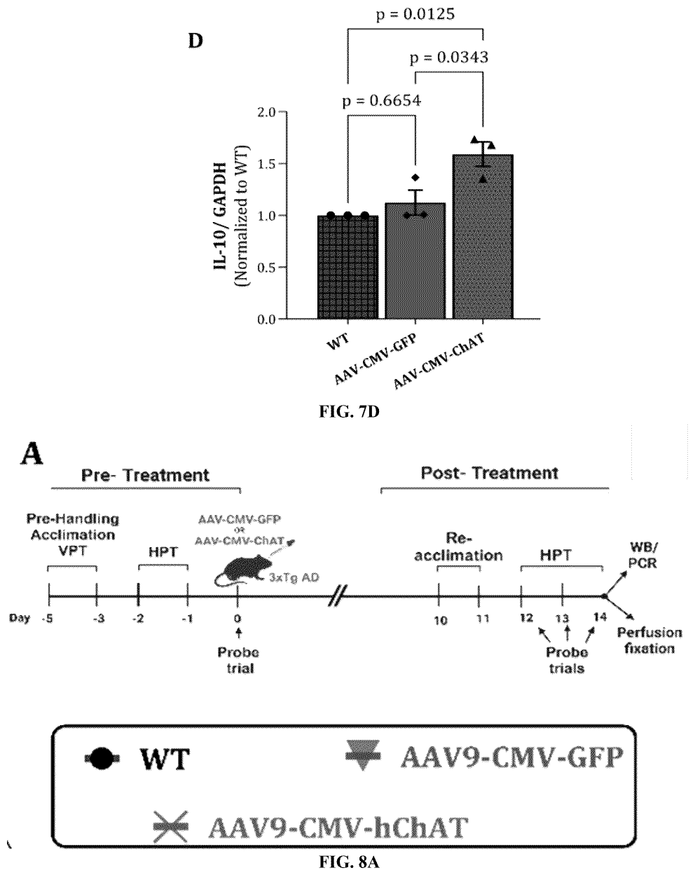

Downstream effects of neuronal and nonneuronal stress responses are modulated in part through the release of cytokines. To assess inflammatory and anti-inflammatory cytokine levels in these young 3×Tg AD mice and to evaluate the modulatory effect of ChAT gene therapy, western blotting was performed on two inflammatory cytokines, TNF-α, and IL-1β, which are involved in disease states, and the anti-inflammatory cytokine, IL-10 (FIG. 7). In the normal physiological state, Tumor Necrosis factor-alpha (TNF-α) plays a protective role through regulating cellular growth and defending cells and intracellular organelles against infection and injuries. However, its role in chronic disease states, such as AD, has not been fully elucidated. In young 3×Tg AD mice, there was a significant difference in the mean OD between the groups (F(2,6)=136.8, p≤0.0001) (FIG. 7A-7B). Further intertreatment analysis revealed a significant increase in the transgenic control group (1.16±0.012) compared with the WT group (1, p=0.0016). ChAT gene therapy significantly attenuated TNF-α activity through its downregulation (0.748±0.028, p≤0.0001). Interleukin-1 beta (IL-1β) is another cytokine that is critically elevated in the disease state and has been implicated in driving chronic inflammatory activity in neurodegenerative disease. Released IL-1β has been associated mostly with microglia and macrophages and is responsible for the production of interleukin-6 (IL-6), which is associated with tangles formation and cognitive impairment in AD. In these young 3×Tg AD mice, ordinary one-way ANOVA did not reveal any significant difference between groups (F(2,6)=4.068, p=0.0765) (FIG. 7). Because there was no significant alteration in Il-1β in in phagocytic microglial activity (CD68+), change in IL-6 was not assessed. Finally, the activity level of IL-10 was analyzed. IL-10 is generally considered an anti-inflammatory cytokine that modulates pro-inflammatory processes such as IL-1β, TNF-α, and IL-6, however, its role and function in driving inflammatory responses in chronic inflammatory conditions such as AD have recently been challenged, and its role in early disease pathogenesis has been further investigated, with studies showing elevated levels of IL-10 in early disease processes in AD mouse models. Herein, there was a significant difference in the mean OD between groups (F(2,6)=10.15, p=0.0119), with multiple comparisons between groups showing no difference between the mean OD values of the transgenic control (1.123±0.1211) and WT (1, p=0.6654) mice (FIGS. 7A and 7D). However, ChAT gene therapy significantly upregulated the activity of IL-10 (1.59±0.1184) indicating the beneficial effect of ChAT gene therapy in modulating early inflammatory responses through the attenuation of proinflammatory processes and the upregulation of anti-inflammatory processes.

### Efficacy of the ChAT Gene in Modulating Learning and Memory in Young 3×Tg AD Mice.

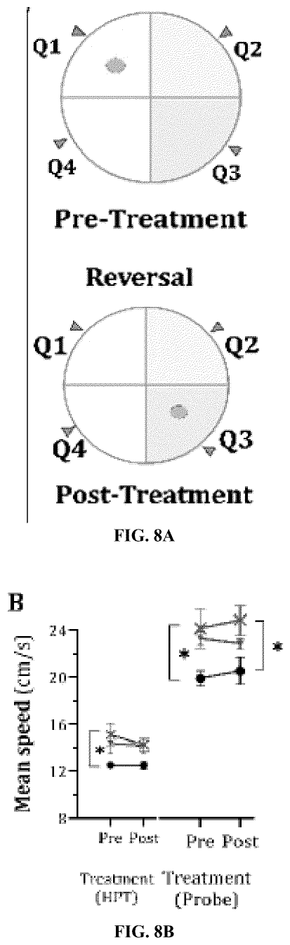

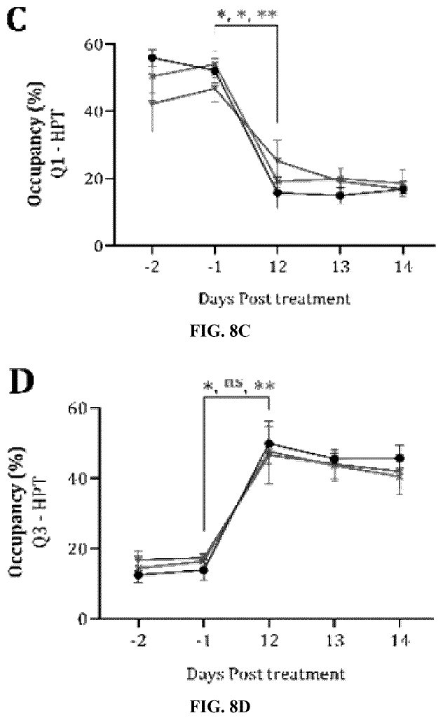

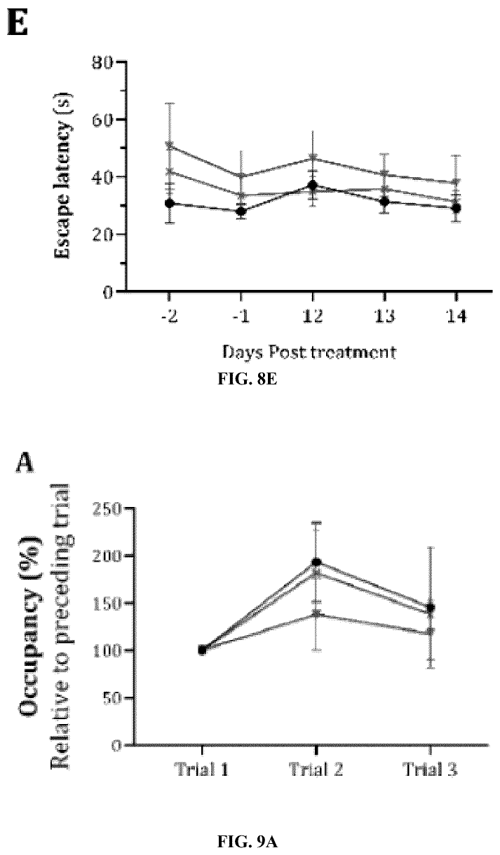

To evaluate early cognitive impairments in 3×Tg AD mice, the Morris water maze (MWM) test was employed. Using the MWM test we evaluated spatial reference and working memory using four visual cues hanging inside the black curtain around the tub, one across each quadrant, and cognitive flexibility and extinction memory by relocating the platform to the opposite quadrant. First, the swimming speed of mice was assessed in each treatment groups to understand their anxiety-like behaviors to swimming and found difference between treatment groups during the HPT (F (2,30)=516 5.903, p=0.0069) as well as during the probe trial (F (2,30)=8.569, p=0.0011) FIG. 8B. Post hoc analysis showed elevated mean speed in 3×Tg AD mice during HPT (Pre-: p=0.0259, Post-: p=0.1749) as well as during probe trial (pre-: 0.0203, post-: 0.0191) compared to WT. There was not an observed treatment effect in this domain. During HPT, each treatment group occupied majority of their experimental time in the target platform quadrants (Q1 pre-treatment and Q3 post-treatment) (FIGS. 8C and 8D). During the pre-treatment phase, when the platform was located at Q1, each mouse showed preference for the target quadrant (Q1) as a function of training days (F (3.306, 49.58)=55.39, p<0.0001), but when the platform was relocated to Q3 during the post-treatment phase, each group of mice spent significantly more time in Q3 (F (2.294, 44.10)=56.50, p<0.0001) and significantly less time in Q1, as shown in FIG. 8 C-D. The WT group exhibited a substantial decrease in Q1 occupancy % (52.1±3.5 Day −1 Vs. 15.8±4.7, Day 12, p=0.0156*), whereas the Q3 occupancy % increased significantly (13.8±2.9, Day −1 Vs. 49.8±6.4, Day 12, p=0.0265*) after platform relocation to Q3. Similarly, the Q1 occupancy percentage decreased considerably in the AAV9-CMV-GFP group (46.7±4.0 day −1 vs. 25.1±6.2, day 12, p=0.0375*), although the Q3 occupancy % increased non-significantly (17.3±1.4, day −1 vs. 46.5±8.2, day 12, p=0.0759, ns) after platform relocation to Q3. In addition, for the AAV9-CMV-ChAT group, the decrease in Q1 occupancy % was substantial (53.9±4.1 day −1 vs. 19.1±1.9, day 12, p=0.0024**), whereas the decrease in Q3 occupancy % significantly increased (16.3±2.2, day −1 vs. 47.6±3.4, day 12, p=0.0017**). However, there was no significant difference between the treatment groups in terms of Q1 occupancy % (F (2,15)=0.3565, p=0.7059) or Q3 occupancy % (F (2,15)=0.0506, p=0.9508). In reference to the time each mouse took to reach the platform (escape latency), the number of training days were generally associated with a decrease in latency time, but there was no significant difference in the escape latency between the treatment groups (F (2,15)=2.166, p=0.1491) FIG. 8E. The escape latency time to the Hidden platform in ChAT gene therapy treated was more closely associated with WT (p=0.9819) than to 3×Tg AD (p=0.6052) mice, implying effect of treatment, even though it did not result in statical significance.

### Efficacy of the ChAT Gene in Modulating Working Memory and Cognitive Flexibility in Young 3×Tg AD Mice.

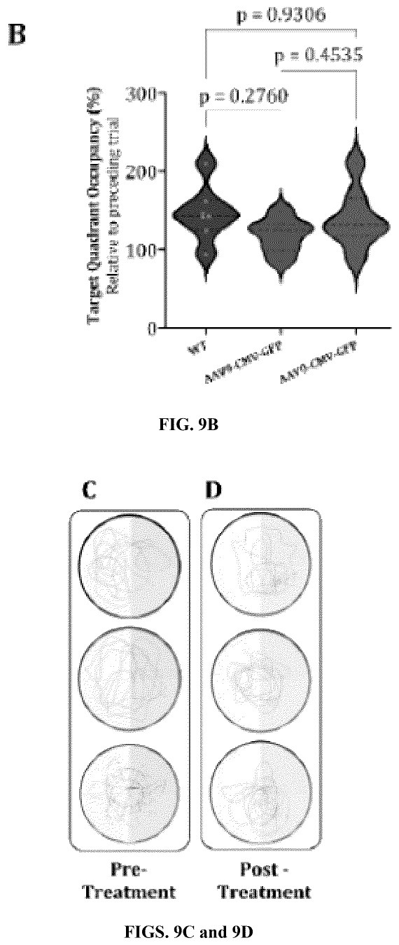

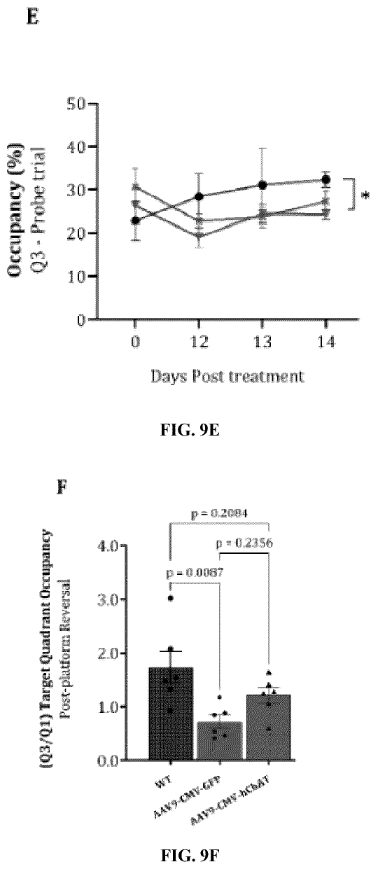

To assess working memory using a water maze, trial-by-trial analyses were conducted on each mouse as they revisited the same platform from different drop locations FIGS. 9A and 9B. On a particular day, 4 trials were conducted per mouse with the platform set in the target quadrant and the mice dropped from all four quadrants. For the purpose of working memory analysis, target quadrant drop location data (e.g., Q3 target quadrant, data with Q3 drop location) were not used because of the proximity of the drop location to the platform. We assumed that the target quadrant occupancy percentage from the first drop was 100% and analyzed the subsequent target quadrant occupancy percentage in comparison to the preceding trial. A value greater than 100% indicated the greater ability of mouse to remember the spatial location of the recently explored pool area and the target quadrant, indicating intact working memory. On the other hand, a value lower than 100% implies decreased working memory. The present disclosure revealed no statically significant difference in working memory between the treatment groups (F(2,18)=1.386, p=0.2755) FIGS. 9A and 9B. When taken together as an average of all trials, there was not an observe significant difference between treatment groups (F(2,18)=1.386, p=0.2755), however, ChAT treated 3×Tg AD mice showed greater intact memory comparable more to that of WT (p=0.9306) than to that of untreated 3×Tg AD mice (p=0.4535) indicating treatment effect FIG. 9B.

To conduct the probe trial, the platform was removed from the tub and allowed each mouse to explore the pool for 60 seconds. A pre-treatment probe trial was conducted on day 0 just before mice were given their respective treatments, and there was not an observed significant difference in the Q1 occupancy percentage between the groups (data not shown). Post-treatment, the platform was relocated to Q3, and a probe trial was conducted on days 12-14 (FIG. 9C-9F). There was not any difference in the target quadrant occupancy percentage as a function of treatment (F (2,15)=1.343, p=0.2908), but Tukey's post hoc analysis revealed a significant depression in target quadrant occupancy in 3×Tg AD mice compared to WT on day 14 (p=0.0131). To assess the impairment in cognitive flexibility and memory extinction associated with early AD, the ration of new target quadrant (Q3) occupancy to that of preceding target quadrant (Q1), and found significant impairment in the ability of 3×Tg AD mice to reacquire the spatial location of the new target quadrant as well as their inability to inhibit a previously learned coordination of the old target (p=0.0087) (FIG. 9F). Treatment group showed improvement in the memory associated with memory extinction and cognitive flexibility as they showed no significant impairment in this domain compared to WT (p=0.2084), even though the improvement was not significant when comparing to GFP treated 3×Tg AD mice (p=0.2356).

### ChAT Gene Therapy Modulates Cell Survival in Part Through Activation of Protein Kinase B (Akt).

To analyze the effect of ChAT gene therapy on modulating cell survival pathways, the relative levels of phosphorylated AKT (protein kinase B, PKB), an effector molecule in the PI3K/AKT pathway that regulates diverse cellular processes, such as cell growth, proliferation, and cell survival was analyzed. Phosphorylated AKT is implicated in pathological conditions such as stroke, AD, Parkinson's disease and diabetes. Other studies suggest that ACh and ChAT may modulate neuroprotective and immunomodulatory effects either through muscarinic acetylcholine receptors, especially M1 receptors, or through nicotinic acetylcholine receptors, both directly and indirectly activating the PI3K/AKT protective pathway. To further elucidate the role of ChAT in modulating cell survival, western blotting was used to analyze the level of phosphorylated AKT in these young 3×Tg AD mice (FIG. 9). Analysis of the relative ratio of phosphorylated AKT (at the Ser473 epitope) to total AKT revealed a significant difference in the mean OD between the treatment groups (F(2,6)=21.26, p=0.0019), and upon further analysis, there was no significant difference between the transgenic control group (1.052±0.06) and the WT group (1, p=0.5981); however, ChAT gene therapy significantly upregulated the amount of phosphorylated AKT (1.32±0.02, p=0.0055). The p-AKT/AKT ratio was also significantly greater in the treatment group than in the WT group (p=0.0022) (10A-10B). The individual levels of phosphorylated AKT was further analyzed and it was found that there was a significant difference between the groups (F(2,6)=9.158, p=0.015) and that the difference was significant between the treatment groups (1.428±0.11) and the WT group (1, p=0.0131) but not between the transgenic control group (1.154±0.051, p=0.0784) (10A and 10C). No significant difference in the mean AKT level was observed between the treatment groups (F(2,6)=1.444, p=0.3076) (10A and 10D).

### DISCUSSION

The present disclosure demonstrated the ability of ChAT gene therapy to protect against early pathological and functional changes in 3×Tg AD mice. ChAT gene therapy reduced the amount of amyloid oligomer deposits, attenuated early phosphorylation of the microtubule-binding domain of tau, protected against mitochondrial dynamics, protected against apoptotic modulators, increased microglial surveillance without inducing activation or inflammation, recruited astrocytes and regulated early inflammatory events. Furthermore, ChAT gene therapy protected young 3×Tg AD mice against functional impairment in the cognitive domain, especially cognitive flexibility and memory extinction, but not against anxiety-like behavior in the Morris water maze test. The present disclosure also demonstrates a protective effect of ChAT that is mediated by activation of the PI3K/AKT signaling pathway.

Other studies have suggested the presence of an extraneuronal cholinergic system in the brain and periphery. Studies have demonstrated the presence of extracellular ChAT, an ACh-synthesizing agent, in human plasma as well as CSF and demonstrated the ability of ChAT to synthesize nonneuronal cells such as astrocytes, microglia and immune cells. Several studies have illustrated the role of ChAT in regulating the innate immune system, inflammation, the activation of high-affinity choline transporters, the regulation of blood pressure, and brain neuroprotection in addition to its critical role in the synthesis of ACh in cholinergic systems. Herein, a ChAT gene therapy was designed using a replication-deficient recombinant AAV9 vector in the presence of a cytomegalovirus (CMV) promoter to deliver ChAT gene therapy to the CNS. To investigate the efficacy of ChAT gene therapy, ChAT gene therapy was delivered to 3×Tg AD mice via one-time eye drop delivery. It was demonstrated that the delivered gene therapy reached brain regions, as measured by the expression levels of transgene mRNA and protein products.

3×Tg AD mice exhibit plaque and tangle pathology as well as cognitive decline with age, and these pathologies recapitulate human AD. Many studies have reported age-related biochemical changes in 3×Tg AD mice, which demonstrated extracellular deposits of plaques within the first six months as well as intracellular accumulation of misfolded tau protein and eventually tangles by 12 months, and these findings have been confirmed by subsequent studies from other laboratories. In 3×Tg AD mice, intracellular deposition of Aβ peptides precedes extracellular deposition, and intracellular Aβ has been observed in the hippocampus and cortex as well as in the amygdala as early as 2 months. Herein, early pathological changes were revealed in 4-month-old 3×Tg AD mice, including changes in the levels of Aβ oligomers and phosphorylated tau protein. There are contradictory reports regarding the age of onset at which Aβ plaques and tangles are observed in 3×Tg AD mice, but studies have consistently shown elevated monomer and oligomer levels of soluble Aβ at early timepoints, starting at 3 months. In ˜4-month-old mice, distinct Aβ₄₂ oligomeric bands at approximately 45 kDa (clusters of 10 Aβ₄₂ monomers) were observed and 3×Tg AD mice had elevated levels of Aβ₄₂ compared to those in age-matched WT mice. Apart from Aβ₄₂, elevated levels of tau phosphorylation in the microtubule-binding domain of tau at threonine 231 (p-tau231) but not at threonine 181 (p-tau181) were also observed. The microtubule-binding domain of the tau protein is responsible for the binding of tau to microtubules, and dysfunction of this functional domain has been implicated in AD. Studies have reported elevated levels of phosphorylated tau protein in 3×Tg AD mice as early as 4 months, and p-Thr231 has been reported to be elevated in AD patients even before the appearance of classical amyloid pathology. The results herein support the use of young 3×Tg AD mice to study early pathological changes in AD. Furthermore, no alterations in the phosphorylation of another widely used tau epitope, p-Thr181 was observed, that are correlated with cognitive deficits.

Oxidative stress is one of the major pathological features of AD and is reported to be a crucial contributor to its progression. Oxidative stress is driven by an imbalance in the number of free radicals such as OH, OH, O₂, NO, and H₂O₂ relative to antioxidant levels, resulting in disruption of cellular processes and eventual cell death. The major source of these intracellular free radicals is mitochondria. Mitochondria produce free radicals and reactive oxygen species during cellular respiration, especially during oxidative phosphorylation in the inner mitochondrial matrix. In physiologically healthy individuals, the generated free radicals are balanced by antioxidants such as superoxide dismutase and glutathione. However, during mitochondrial damage and dysfunction, mitochondria produce excessive free radicals that leak to the cytoplasm as well as to the extracellular space, where they drive toxic events. Studies have reported excessive bioenergetic dysregulation and disruption of glucose metabolism in individuals with increased AD risk, such as individuals with Down syndrome and mild cognitive impairment. Furthermore, multiple studies have reported bioenergetic dysregulation and disruption in different parts of the electron transport chain in AD patients via fluorodeoxyglucose (18F)-PET (FDG-PET) scanning analysis. Additionally, mitochondrial and bioenergetic dysfunctions have been reported in animal models of AD, including a recent study that reported a deficit in mitochondrial bioenergetics in 2- to 3-month-old female 3×Tg AD mice. 3×TgAD mice reportedly exhibit impaired glucose metabolism as early as 5 weeks, along with metabolic abnormalities, including impaired GLUT3 translocation to the plasma membrane, and these abnormalities progressively lead to a reduction in glucose uptake in brain regions such as the amygdala, entorhinal cortex, and hippocampus. Furthermore, DNA and mitochondrial proteins involved in energy metabolism as well as in synapses, apoptosis and oxidative stress were impaired in these mice. Another study reported alterations in brain ATP levels along with alterations in the phospholipid composition of the synaptic mitochondrial membrane. Furthermore, herein it was revealed that early bioenergetic dysfunction in 3×Tg AD mice is associated with impairment of the astrocytic biosynthetic pathway, leading to synaptic plasticity and behavioral deficits. In line with these above observations, it was herein demonstrated dysregulation of mitochondrial fusion and fission along with alterations in mitophagy, indicating mitochondrial damage and active mitophagy. Furthermore, when comparing the ratio of fusion to fission, there were significantly elevated fission processes, indicating malfunctioning mitochondrial processes.

Mitochondria maintain the dynamic nature of cellular processes such as ATP production, fatty acid synthesis, ROS generation, oxidative phosphorylation, and Ca²⁺ hemostasis through processes such as fission, fusion, and mitophagy. Disruption of fission, fusion or mitophagy disrupts mitochondrial dynamics and may result in excessive production of free radicals, resulting in oxidative stress. Mitochondrial fission, the process by which a mitochondrion splits into two parts, is mediated by a cytoplasmic GTPase protein called Drp1, whereas fusion is the process by which two different mitochondria fuse into one and is mediated in part through Opa1, an inner mitochondrial membrane molecule that plays a critical role in the fusion of inner mitochondrial membranes. Both Opa1 mutant/double knockout mice and Drp1 knockout mice exhibit embryonic lethality, indicating the critical role of both of these proteins in mitochondrial function. The results indicate that these young mice have dysregulated fission and fusion processes. Since Opa1 plays an important role in maintaining mitochondrial DNA, bioenergetics, oxidative phosphorylation, and calcium homeostasis as well as in the release of cytochrome C, abnormalities in the Opa1 protein, in addition to elevated fission, could promote endoplasmic calcium overload, apoptosis dysregulation and downstream stress signaling. One downstream effect of the dysregulation of fusion and fission is the overactivation of mechanisms to eliminate damaged mitochondria, known as mitophagy. Mitochondrial dysfunction or dysregulation can activate mitophagy, which is regulated in part through the binding of adaptor molecules such as p62 to mitochondria-tagged ubiquitin chains. Binding of p62 to mitochondria serves as a signal for removal, at which point p62 interacts with microtubule-associated protein 1 α/β light chain 3 (MAP LC3α/β). Binding with MAP LC3α/β initiates autophagosome formation and mitophagy. Although no activated mitophagy markers, such as p62 and MAP LC3α/β was observed, ChAT gene therapy activated these processes, showing that the protective function of ChAT could be through activating mitophagy processes and removing damaged mitochondria. Mice treated with ChAT also exhibited improvements in mitochondrial dynamics through the increase in the expression of protective fusion proteins as well as through the inhibition of Drp1 activity. The brain tissues were further analyzed to investigate whether general autophagy was affected, but no significant changes in overall autophagic biomarkers, such as Beclin-1, were observed, indicating that the autophagy process was restricted to mitochondria at this stage (mitophagy). Mitochondrial permeabilization and the cytoplasmic release of free radicals and Ca²⁺ can lead to Ca²⁺ overload in the ER, resulting in disruption of the unfolded protein response and ER stress. No significant changes in biomarkers associated with endoplasmic reticulum stress, such as GRP78 and CHOP, were observed, which are central to the ER-associated stress response, indicating that during this stage of disease progression, mitochondrial dysfunction does not affect other intercellular complexes.

Damaged or dysregulated mitochondria result in the permeabilization of the outer mitochondrial membrane, resulting in the release of cytochrome c to the cytoplasm, where it initiates intrinsic apoptosis signaling, leading to cellular apoptosis. In the cytoplasm, cytochrome-c binds to apoptotic protease activating factor 1 (Apaf-1), forming the apoptosome that activates caspase-9 and eventually the executioner caspase-3. The Bcl-2 family of proteins plays an important role in regulating apoptosis signaling. Pro-apoptotic proteins such as Bax, Bak, Bim and Puma promote cellular death, whereas anti-apoptotic proteins such as Bcl-2 and Bcl-x promote cell survival. Pro-apoptotic proteins such as Bax promote mitochondrial outer membrane permeabilization in addition to the activation of other pro-apoptotic proteins such as Bak, whereas anti-apoptotic proteins such as Bcl-2 promote cell survival by interacting with and neutralizing Bax, resulting in the stabilization of the outer mitochondrial membrane and the inhibition of cytochrome c release. Bcl-2 can also inhibit apoptosis through the sequestration of apoptotic proteins such as Bim and Puma, preventing the activation of Bax and Bad. The results herein indicated that young 3×Tg AD mice were in the early phase of dysregulation of apoptotic processes, as evidenced by a nonsignificant decrease in the level of the protective protein Bcl-2 and a nonsignificant increase in the level of the apoptotic protein Bax. However, when comparing their relative ratios, the result was a marked increase in apoptotic processes, implying an impairment in the cell survival pathway. Importantly, in transgenic mice treated with ChAT, both the ratio of Bcl-2 and Bax and the amount of Bcl-2 and Bax tended to increase, indicating the beneficial effect of ChAT gene therapy.

While acute inflammation is the body's normal defense mechanism against injury and pathogens, imbalances exist between pro- and anti-inflammatory biomarkers in AD patients, leading to chronic inflammation and progressive AD pathology. Chronic inflammation in the CNS is driven mainly through the activity of microglia and astrocytes. Ionized calcium binding adaptor molecule 1 (Iba1) is a microglial biomarker that is expressed at optimal levels in ramified microglia with elongated processes. Iba1 has been consistently used as a pan microglial marker to indicate microglial motility and migration ability. On the other hand, cluster of differentiation 68 (CD68), a transmembrane glycoprotein that is expressed by activated microglia with an amoeboid morphology, has been widely observed in chronic disease conditions such as AD, PD, and other neurodegenerative diseases. Microglia, monocytes and tissue macrophages that express CD68 have consistently been associated with phagocytic activity. In AD patients, postmortem analysis of the cerebral cortex revealed a correlation between disease severity and microglial function. While activated and phagocytic microglia (CD68+ levels) increase with disease severity, microglial mobility and surveillance ability (Iba1+) decrease, indicating a relationship between disease progression, phagocytic activity and microglial surveillance ability. In fact, Iba1+ microglia are now considered to be associated with pathologically healthy individuals as well as early microglial activity during the moderate activation of microglia, as evident from their expression by microglia capable of membrane ruffling and ramification but not with chronically activated microglia. Even in studies in which Iba1 was expressed in severe AD patients, no difference was found in the number of Iba1+ microglia compared to that in healthy controls, showing that microglial activation and phagocytic activity are a result of changes in morphology instead of an increase in the number of microglia. This explains the findings from multiple human studies that have revealed elevated numbers of Iba1+ microglia in early disease states but not in advanced disease states, where elevated numbers of CD68+ microglia have been observed. In a study assessing the microglial phenotype in deep subcortical lesions in patients, it was previously found that elevated levels of CD68⁺ and MHC-II⁺ microglia along with elevated levels of Iba1⁺ microglia in “normal-appearing” white matter but significantly reduced levels of Iba1⁺ microglia in patients with deep subcortical lesions that also showed elevated numbers of CD68+ microglia. In another study, elevated levels of the inflammatory microglial marker CD68 and reduced levels of Iba1 with increasing severity of dementia, and these findings have been confirmed in other AD patient studies.

Animal models of Alzheimer's disease have been used to further elucidate the role of microglia in AD pathogenesis. A study conducted to assess the role of microglia-mediated inflammation demonstrated elevated levels of microglial proliferation and activation in different brain regions of 2-month-old 3×Tg AD mice. Furthermore, herein it was revealed that morphological changes in microglia, including smaller and more complex cells. Other studies on age-related microglial and astrocytic changes in 3×TgAD mice have shown an age-dependent increase in activated microglia in the HPC and the entorhinal cortex starting at six months of age. Herein, there was not an observation of any significant change in the number of CD68+ microglia or the Iba1+ microglial population, but the relative ratio of Iba1 to CD68 was significantly increased, indicating early microglial migration and increased surveillance activity, indicating early pathological events in these mice. Considering that these mice exhibit mitochondrial dysfunction, activated mitophagy, increased Aβ42 oligomer levels without observable amyloid plaque pathology, and a normal state of ER and autophagy markers, the elevated level of Iba1+ microglia may indicate a heightened state of microglial alertness without activating the inflammatory response to minor fluctuations in the brain microenvironment. This is supported by studies in very young 3×Tg AD mice in which an increased number of smaller and more complex microglia were observed at 2 months. Furthermore, their observation of more complex and smaller microglia shows hyper ramified microglia, which can explain the elevated level of Iba1. Furthermore, GFAP levels were found to be significantly decreased in these mice, and this finding is supported by other studies that have reported age-related reductions in the number and volume of astrocyte processes up to 12 months, when the number of astrocytes starts to increase critically. A reduction in the number of astrocytes has also been reported in AD patients during the early disease process. The role of astrocytes in maintaining the brain homoeostatic system, including metabolic support, BBB regulation, and neuronal support, is well documented. In neuronal systems, they play critical roles in the maintenance of extracellular potassium and glutamate and hence are critical for neuronal excitability and excitotoxicity. While chronic injuries inducing reactive astrogliosis and acute inflammation are well characterized, the role of reduced astrocytic complexities and/or quantity in driving the early disease state is not fully understood. ChAT gene therapy showed promising results in restoring GFAP expression in 3×Tg AD mice, showing that ChAT has a protective effect on the physiological function of GFAP.

AD animal model studies have revealed a fascinating shift in the patterns of inflammatory cytokines during AD progression, with an early phase of dampened cytokine activity followed by excessive activation during the advanced stage of the disease. Understanding the events that lead to this transition may shed light on the pathophysiology of AD, although much remains unknown. The activation of glial cells, especially microglia and astrocytes, which are consistently observed in the vicinity of amyloid beta plaques in AD pathology, has been shown to drive chronic inflammation through the release of inflammatory cytokines such as IL-1β, TNF-α, and IL-6, along with toxic products such as reactive oxygen species and nitric oxide, which further exacerbate AD pathology. IL-1β is widely considered a master regulator of the brain inflammatory cascade because it regulates the expression of proinflammatory cytokines such as IL-6 and TNF-α. In AD, IL-1β regulates APP processing through increased γ-secretase activity, leading to elevated levels of insoluble amyloid beta, and the accumulated plaque eventually stimulates microglia to produce more TNF-α and IL-1β in a feed forward loop, creating a cascading event exacerbating AD pathology. Elevated levels of IL-1β are involved in the production of another inflammatory cytokine, IL-6, which plays an important role in the hyperphosphorylation of tau through its modulation of cyclin-dependent kinase 5 (CDK5). An elevated level of IL-6 has been shown to be closely associated with cognitive decline in AD patients and has been detected in the CSF and blood serum of patients who exhibit amyloid plaques. Studies have shown elevated levels of IL-1β in the dentate gyrus and pyramidal neurons of the hippocampus as well as in the prefrontal cortex in early AD patients, and these levels continue to increase with disease progression. Similarly, TNF-α plays a critical role in the initiation and regulation of cytokines during Aβ-induced inflammation through the activation of the transcription factor NF-kB. Furthermore, TNF-α has been shown to increase the production of Aβ through the upregulation of BACE 1 and γ-secretase production. Studies have shown elevated levels of TNF-α in the plasma and brain tissues of AD patients as well as in aged mouse models. As eluded previously, studies have shown the fascinating phenomenon of an inflammatory cytokine profile with an initial reduction in inflammatory cytokine levels at the onset of disease (as Aβ starts to accumulate) that eventually increases chronically in the later stage of disease progression. 3×Tg AD mice have also been shown to exhibit this phenomenon. In a previous study, a general reduction in the levels of inflammatory cytokines such as IL-1β, TNF-α, IL-6, and IL-18 was seen at 3 months of age; these levels either tended toward an elevated state or were found to be significantly elevated by 9 months, at a point when these mice exhibited prominent amyloid pathology. Herein, substantially elevated levels of TNF-α were revealed but no significant changes in IL-1β levels in whole-brain extracts. Furthermore, no change in the levels of the anti-inflammatory interleukin IL-10 was seen. It is well documented that Aβ can stimulate activated microglia to produce TNF-α and IL-1β. However, the present disclosure did not detect activated microglial or astrocytic biomarkers (CD68 or GFAP for astrocytes), indicating either the involvement of microglia and astrocytes expressing other biomarkers or the activation of TNF-α through other means, such as mitochondrial or neuronal stress-induced signaling pathways. Importantly, ChAT gene therapy demonstrated a modulatory effect on the activity of these cytokines by decreasing the amount of TNF-α and through the significant upregulation of the anti-inflammatory interleukin IL-10.

AD patients exhibit a progressive decline in cognitive function characterized by early impairments in associative learning, working memory, and episodic memory followed by further dysfunction in spatial learning and memory, cognitive flexibility, neuropsychiatric changes, global memory decline and dementia. Individuals with 3×Tg-AD manifest these cognitive abnormalities in an age-dependent manner, with impairments in associative learning, spatial working memory, reference memory, and cognitive flexibility observed within the first 6 months. Herein, 4-month-old 3×Tg AD mice did not exhibit significant impairment in spatial reference memory; however, working memory was significantly impaired compared with that of age-matched nontransgenic wild-type mice. There was no difference in escape latency in the HPT phase even though 3×Tg AD mice consistently tended toward greater escape time in the pool, indicating potential impairment of reference memory. Furthermore, similar patterns were observed in working memory, with no significant difference in trial-over-trial target quadrant occupancy between genotypes, but 3×Tg AD mice also tended toward impairment in this domain. However, these young mice exhibited impairments in other behavioral domains. Elevated swimming speed was observed in 3×Tg AD mice compared to wild-type controls during both HPT and probe trials, indicating anxiety-like behavior. Furthermore, in the reversal phase of the MWM test, there was a significant difference in the percentage occupancy of the target quadrant between the WT and 3×Tg AD mice on day 14. In addition, 3×Tg AD mice had significant impairments in memory extinction associated with the previously acquired spatial location of the hidden platform in addition to the acquisition of new memories on the spatial orientation of the new platform location, implying impaired cognitive flexibility as well as memory consolidation. Importantly, compared with WT mice, transgenic mice treated with ChAT did not show a significant impairment in the ability to acquire spatial information about the new platform location alongside memory extinction regarding the preceding platform location. Even during working memory trials, ChAT treatment had some nonsignificant beneficial effects on improving trial-over-trial target quadrant occupancy, even though it had no effect on modulating anxiety-related live behavior (swim speed). In summary, 4-month-old 3×Tg AD mice did not exhibit significant impairment in spatial working or reference memory, but they exhibited anxiety-like behavior and impaired cognitive flexibility and memory consolidation, as measured during platform reversal training. Importantly, ChAT gene therapy protected 3×Tg AD mice from these deficits, except for anxiety-like behavior.

AKT (also known as protein kinase B or PKB) is a downstream effector of phosphoinositide-3-kinase (PI3K) that is implicated in metabolic regulation, cell survival and proliferation, cell signaling, stress responses, and embryonic development, among other cellular processes, and dysregulation of AKT is implicated in many disease pathologies, including type 2 diabetes and neurodegenerative diseases. PI3K-AKT signaling is a major cell survival pathway that regulates apoptosis, cognitive function, autophagy, glucose metabolism, oxidative stress, long-term potentiation (LTP), and inflammation, among other processes. Downregulation of AKT or impairment of the PI3K-AKT signaling pathway is implicated in mitochondrial dysfunction, leading to a surge in free radicals as well as Ca²⁺ overload, promoting the translocation of cytochrome c and apoptosis inducible factors (AIFs) from the inner mitochondrial membrane to the cytoplasm and ultimately leading to apoptosis. Along with aberrant mitochondrial dynamics and increased apoptosis and oxidative stress, AKT attenuation has been reported to be involved in impaired GLUT translocation, leading to metabolic abnormalities. Studies have shown a significant reduction in p-AKT in 3×Tg AD mice. Interestingly, 4-month-old 3×Tg AD mice showed a nonsignificant increase in the level of phosphorylated AKT and a slight increase in the level of baseline AKT, showing that the activation of p-AKT may be associated with the protective mechanism of the neuronal microenvironment. Importantly, ChAT gene therapy significantly increased the level of phosphorylated AKT and the p-AKT-to-AKT ratio. The significance of ChAT therapy could be to activate the AKT pathway early in the disease process, which could counteract any possible damage from energy dysfunction, alterations in mitochondrial dynamics and protection against downstream stress-related apoptosis.

The biomarkers downstream of the PI3K-AKT pathway were further analyzed to investigate whether the protective effects of ChAT gene therapies are mediated through the AKT pathway. The results herein provide convincing evidence that ChAT or ACh activate the protective AKT signaling pathway. Some dysregulation of cell survival pathways and apoptosis as well as the release of inflammatory cytokines were found. The relative ratio of the prosurvival and anti-apoptotic biomarker Bcl-2 to the apoptotic biomarker Bax was measured and it was found that the ratio of BCl-2/Bax was slightly decreased. This ratio was concurrent with a slight decrease in the level of Bcl-2 and an increase in the level of Bax. ChAT gene therapy reversed these changes even though the improvements were not significant. These changes in cell survival markers in combination with disturbances in mitochondrial dynamics could be indicative of oxidative stress, which could be a result of bioenergetic abnormalities that have been documented in very young 3×Tg AD mice.

Owing to these observations, therapies that can preemptively activate the PI3K-AKT pathway could change the trajectory of AD. Here, it was demonstrated that while the p-AKT/AKT ratio was not significantly altered in 3- to 4-month-old 3×Tg-AD mice, ChAT gene therapy significantly activated the p-AKT pathway, leading to significant reversal of mitochondrial dysfunction and relative attenuation of apoptosis and potentially altering the disease progression timeline, which could become increasingly significant as the disease progresses.

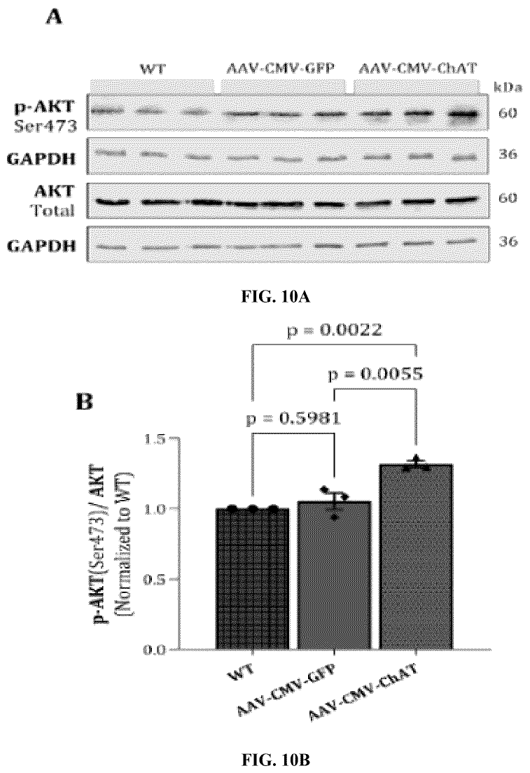

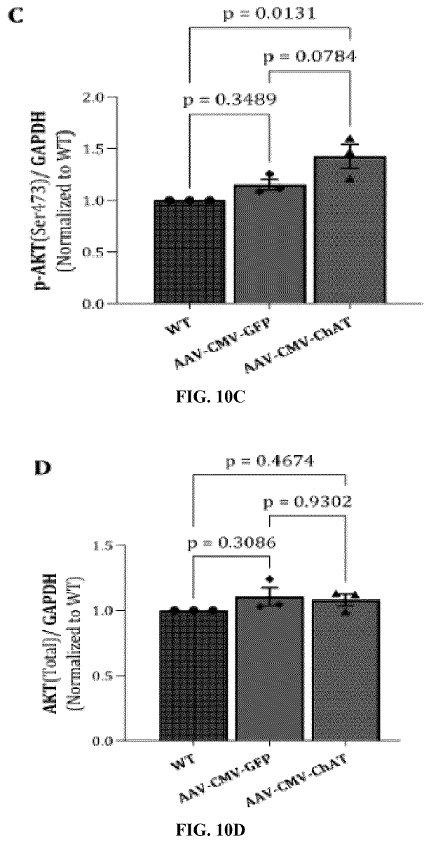

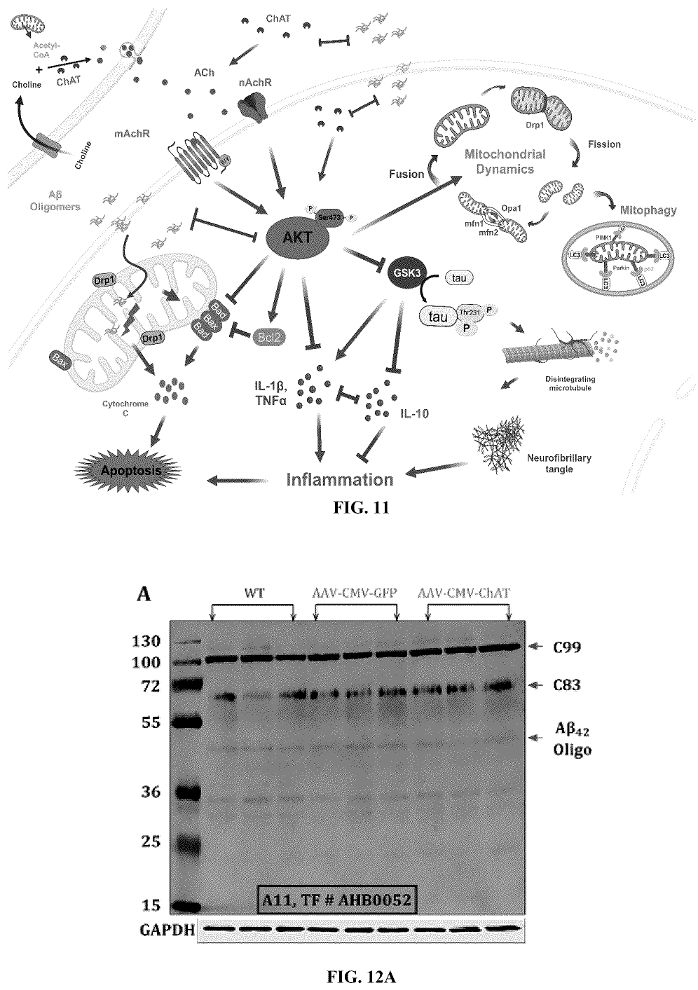

In summary, the physiological events leading from the upregulation of ChAT and ACh to the neuroprotection shown by ChAT gene therapy can be described as follows FIG. 10. Delivered gene therapy produces functional hChAT protein in the CNS of mice. Upregulated hChAT biosynthesizes ACh in presynaptic cells. ACh is released to the synaptic terminal along with some ChAT (the mechanism of which is still unknown). ACh in the synaptic terminal acts on muscarinic acetylcholine receptors (mAChRs) as well as nicotinic acetylcholine receptors (nAChRs) and activates phosphoinositide 3-kinase (PI3K) and the downstream effector protein kinase B (AKT). Activated AKT exerts its protective influence on various cellular processes-A) modulating mitochondrial dynamics through the regulation of fission, fusion and mitophagy; B) inhibiting the release of cytochrome C from mitochondria through the downregulation of fission, the promotion of fusion, the inhibition of the formation of the Bax-Bad complex and the subsequent permeabilization of the outer mitochondria; C) modulating the activity of microglia, astrocytes, and neurons to release inflammatory cytokines; and D) membrane phosphorylation and inactivation of GSK3, leading to the inhibition of tau hyperphosphorylation. This proposed mode of action of ChAT is supported by the data herein. With respect to mitochondrial dynamics, there was significant upregulation of Opa1 and p62 in addition to a significant reduction in Drp1. Increasing Opa1 along with a reduction in the level of Drp1 is indicative of the activation of protective mechanisms in mitochondria. In addition, p62- and LC3-mediated activation of mitophagy is protective in the neuronal microenvironment, especially in the early disease process. Impaired insulin signaling, bioenergetic failure, and mitochondrial dysfunction are hallmark features of AD, and they precede AD pathology. Importantly, these impairments have been associated with dysfunction of the PI3K-AKT signaling pathway. Furthermore, dysfunction in mitochondrial dynamics, including fission mediated by Drp1, can lead to the release of cytochrome C and downstream apoptosis. It was demonstrated that ChAT gene therapy can modulate the relative activity of Bcl-2 and Bax by upregulating the anti-apoptotic protein Bcl-2, which has been implicated in its ability to neutralize the proapoptotic protein Bax, inhibiting it from permeabilizing the mitochondrial outer membrane and eventually releasing cytochrome C, as well as inhibiting the activity of Bax directly. Additionally, studies have reported that this pro-survival modulation of ChAT and ACh is attributed to the activation of the PI3K-AKT pathway by AChRs. In addition, studies have suggested the involvement of nAChRs that activate the PI3K-AKT pathway in neuronal protection triggered by amyloid beta accumulation. In addition, it was demonstrated that ChAT gene therapy modulated the activity of GFAP and the relative ratio of activated to motile microglia and significantly reduced the level of TNF-α and increased the level of protective IL-10, showing that the neuroprotective effect of ChAT and this activity of AChRs was attributed to the PI3K-AKT pathway. Additionally, the PI3K-AKT pathway is implicated in the suppression of the GSK-3β-mediated phosphorylation of tau protein. The PI3K-AKT pathway modulates cellular functions such as glucose metabolism, cellular transport and apoptosis through its inhibitory regulation of GSK-3β. Herein, it was demonstrated that ChAT gene therapy modulated the relative phosphorylation of tau at the epitope threonine 231 through activation of the PI3K-AKT pathway. These findings indicate the ChAT gene therapy as a therapeutic approach for early AD patients, as its activity is linked to the activation of the protective PI3K-AKT pathway.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### TABLES

TABLE 1
List of antibodies used.

| Primary | Company | Host/Isotype | | Concen- |
|---|---|---|---|---|
| Antibody | Catalog# | Class | kDa | tration |
| Anti - IL-10 | Santa Cruz | Mousse/IgG | 22 | 1/250 |
| | Biotechnology SC- | monoclonal | | |
| | 8438 | | | |
| Anti- TNF-α | Rockland | Rabbit/IgG | 20 | 1/500 |
| | 210-401-321 | monoclonal | | |
| Anti- CHOP | Santa Cruz | Mouse/IgG | 30 | 1/250 |
| GADD153 | Biotechnology SC- | monoclonal | | |
| | 7351 | | | |
| Anti- Bcl-2 | TF Scientific | Rabbit/IgG | 26 | 1/500 |
| | PA5-20068 | Polyclonal | | |
| Anti- p-Thr181 | Novus Biologicals | Rabbit/IgG | 47 | 1/500 |
| (p-tau) | NB100-82245 | Polyclonal | | |
| Anti- p-Thr231 | Novus Biologicals | Rabbit/IgG | 50 | 1/500 |
| (p-tau) | NB100-82249 | Polyclonal | | |
| Anti- IL-1B | Cell signaling | Mouse/IgG | 31 | 1/500 |
| | 12242S | monoclonal | | |
| Anti- Iba1 | FIJIFILM Wako | Rabbit/ | 15 | 1/500 |
| | 019-19741 | Polyclonal | | |
| Anti- CD68 | Novus Biologicals | Mouse/IgG | 68 | 1/500 |
| | NB600-985 | monoclonal | | |
| Anti- A11 Oligo | TF Scientific | Rabbit/IgG | 44 | 1/1000 |
| | AHB0052 | Polyclonal | | |
| Anti- GFAP | Cell signaling | Rabbit/IgG | 50 | 1/500 |
| | 80788S | monoclonal | | |
| Anti- hChAT | Millipore Sigma | Mouse/IgG1 | 69 | 1/1000 |
| 1.B3.9B3 | MAB5270 | Monoclonal | | |
| Anti- p-AKT | Cell signaling | Rabbit/IgG | 60 | 1/2000 |
| Ser473 | 4060S | monoclonal | | |
| Anti- AKT (Pan) | Cell signaling | Rabbit/IgG | 60 | 1/1000 |
| C67E7 | 4691S | monoclonal | | |
| Anti- GRP78 Bip | Abcam | Rabbit/IgG | 75 | 1/1000 |
| | Ab21685 | Polyclonal | | |
| GAPDH | Cell signaling | Rabbit/IgG | 36 | 1/4000 |
| | 5174S | monoclonal | | |
| GAPDH | Cell signaling | mouse/IgG | 36 | 1/4000 |
| | 97166S | monoclonal | | |
| Opa1 | Abcam | Rabbit/IgG | 112 | 1/1000 |
| | Ab157457 | monoclonal | | |
| Drp1 | Abcam | Rabbit/IgG | 83 | 1/2000 |
| | Ab184247 | Monoclonal | | |

TABLE 2
qPCR Primer Design

| | Accession | Primer sequence | Primer | Amplicon |
|---|---|---|---|---|
| Gene Name | No. | (5′ à 3′) | size | size (bp) |
| Human-choline | BC130615 | F: | 22 | 82 |
| acetyltransferase | | TGTCATTAATTTCCGCCGTCTC | | |
| (hChAT) | | (SEQ ID NO: 3) | | |
| | | R: GTCCTCGTTGGAAGCCATTT | 20 | |
| | | (SEQ ID NO: 4) | | |
| Glyceraldehyde | BC145810 | F: | 23 | 207 |
| -3-phosphate | | CACTCACGGCAAATTCAACGGC | | |
| dehydrogenase | | A (SEQ ID NO: 5) | | |
| (GAPDH) | | R: | 23 | |
| | | ATGACCCTTTTGGCTCCACCCTT | | |
| | | (SEQ ID NO: 6) | | |

SEQUENCES
1. SEQ ID NO: 1-replication-deficient recombinant
adeno-associated virus (AAV) vector with ChAT
CCTGCAGGCAGCTGCGCGCTCGCTCGCTCACTGAGGCCGCCCGGGCGTCGGGCGACCTTTGGTCG
CCCGGCCTCAGTGAGCGAGCGAGCGCGCAGAGAGGGAGTGGCCAACTCCATCACTAGGGGTTCCT
GCGGCCAATTCAGTCGATAACTATAACGGTCCTAAGGTAGCGATTTAAATACGCGCTCTCTTAAG
GTAGCCCCGGGACGCGTCAATTGCATGAAGAATCTGCTTAGGGTTAGGCGTTTTGCGCTGCTTCG
CGATGTACGGGCCAGATATACGCGTTGACATTGATTATTGACTAGTTATTAATAGTAATCAATTA
CGGGGTCATTAGTTCATAGCCCATATATGGAGTTCCGCGTTACATAACTTACGGTAAATGGCCCG
CCTGGCTGACCGCCCAACGACCCCCGCCCATTGACGTCAATAATGACGTATGTTCCCATAGTAAC
GCCAATAGGGACTTTCCATTGACGTCAATGGGTGGAGTATTTACGGTAAACTGCCCACTTGGCAG
TACATCAAGTGTATCATATGCCAAGTACGCCCCCTATTGACGTCAATGACGGTAAATGGCCCGCC
TGGCATTATGCCCAGTACATGACCTTATGGGACTTTCCTACTTGGCAGTACATCTACGTATTAGT
CATCGCTATTACCATGGTGATGCGGTTTTGGCAGTACATCAATGGGCGTGGATAGCGGTTTGACT
CACGGGGATTTCCAAGTCTCCACCCCATTGACGTCAATGGGAGTTTGTTTTGGCACCAAAATCAA
CGGGACTTTCCAAAATGTCGTAACAACTCCGCCCCATTGACGCAAATGGGCGGTAGGCGTGTACG
GTGGGAGGTCTATATAAGCAGAGCTCTCTGGCTAACTAGAGAACCCACTGCTTACTGGCTTATCG
AAATTAATACGACTCACTATAGGGAGACCCAAGCTGGCTAGCGTTTAAACTTAAGCTTGGTACCG
AGCTCGGATCCACTAGTCCAGTGTGGTGGAATTCTGCAGATTGATTATGGCAGCAAAAACTCCCA
GCAGTGAGGAGTCTGGGCTGCCCAAACTGCCCGTGCCCCCGCTGCAGCAGACCCTGGCCACGTAC
CTGCAGTGCATGCGACACTTGGTGTCTGAGGAGCAGTTCAGGAAGAGCCAGGCCATTGTGCAGCA
GTTTGGGGCCCCTGGTGGCCTCGGCGAGACCCTGCAGCAGAAACTCCTGGAGCGGCAGGAGAAGA
CAGCCAACTGGGTGTCTGAGTACTGGCTGAATGACATGTATCTCAACAACCGCCTGGCCCTGCCT
GTCAACTCCAGCCCTGCCGTGATCTTTGCTCGGCAGCACTTCCCTGGCACCGATGACCAGCTGAG
GTTTGCAGCCAGCCTCATCTCTGGTGTACTCAGCTACAAGGCCCTGCTGGACAGCCACTCCATTC
CCACTGACTGTGCCAAAGGCCAGCTGTCAGGGCAGCCCCTTTGCATGAAGCAATACTATGGGCTC
TTCTCCTCCTACCGGCTCCCCGGCCATACCCAGGACACGCTGGTGGCTCAGAACAGCAGCATCAT
GCCGGAGCCTGAGCACGTCATCGTAGCCTGCTGCAATCAGTTCTTTGTCTTGGATGTTGTCATTA
ATTTCCGCCGTCTCAGTGAGGGGGATCTGTTCACTCAGTTGAGAAAGATAGTCAAAATGGCTTCC
AACGAGGACGAGCGTTTGCCTCCAATTGGCCTGCTGACGTCTGACGGGAGGAGCGAGTGGGCCGA
GGCCAGGACGGTCCTCGTGAAAGACTCCACCAACCGGGACTCGCTGGACATGATTGAGCGCTGCA
TCTGCCTTGTATGCCTGGACGCGCCAGGAGGCGTGGAGCTCAGCGACACCCACAGGGCACTCCAG
CTCCTTCACGGCGGAGGCTACAGCAAGAACGGGGCCAATCGCTGGTACGACAAGTCCCTGCAGTT
TGTGGTGGGCCGAGACGGCACCTGCGGTGTGGTGTGCGAACACTCCCCATTCGATGGCATCGTCC
TGGTGCAGTGCACTGAGCATCTGCTCAAGCACATGACGCAGAGCAGCAGGAAGCTGATCCGAGCA
GACTCCGTCAGCGAGCTCCCCGCCCCCCGGAGGCTGCGGTGGAAATGCTCCCCGGAAATTCAAGG
CCACTTAGCCTCCTCGGCAGAAAAACTTCAACGAATAGTAAAGAACCTTGACTTCATTGTCTATA
AGTTTGACAACTATGGGAAAACATTCATTAAGAAGCAGAAATGCAGCCCTGATGCCTTCATCCAG
GTGGCCCTCCAGCTGGCCTTCTACAGGCTCCACCGAAGACTGGTGCCCACCTACGAGAGCGCGTC
CATCCGCCGATTCCAGGAGGGACGCGTGGACAACATCAGATCGGCCACTCCAGAGGCACTGGCTT
TTGTGAGAGCCGTGACTGACCACAAGGCTGCTGTGCCAGCTTCTGAGAAGCTTCTGCTCCTGAAG
GATGCCATCCGTGCCCAGACTGCATACACAGTCATGGCCATAACAGGGATGGCCATTGACAACCA
CCTGCTGGCACTGCGGGAGCTGGCCCGGGCCATGTGCAAGGAGCTGCCCGAGATGTTCATGGATG
AAACCTACCTGATGAGCAACCGGTTTGTCCTCTCCACTAGCCAGGTGCCCACAACCACGGAGATG
TTCTGCTGCTATGGTCCTGTGGTCCCAAATGGGTATGGTGCCTGCTACAACCCCCAGCCAGAGAC
CATCCTTTTCTGCATCTCTAGCTTTCACAGCTGCAAAGAGACTTCTTCTAGCAAGTTTGCAAAAG
CTGTGGAAGAAAGCCTCATTGACATGAGAGACCTCTGCAGTCTGCTGCCGCCTACTGAGAGCAAG
CCATTGGCAACAAAGGAAAAAGCCACGAGGCCCAGCCAGGGACACCAACCTTGAAATCCCATCCA
GCACAGTGGCGGCCGCTCGAGTCTAGAGGGCCCTTCGAAGGTAAGCCTATCCCTAACCCTCTCCT
CGGTCTCGATTCTACGCGTACCGGTCATCATCACCATCACCATTGAGTTTAAACCCGCTGATCAG
CCTCGACTGTGCCTTCTAGTTGCCAGCCATCTGTTGTTTGCCCCTCCCCCGTGCCTTCCTTGACC
CTGGAAGGTGCCACTCCCACTGTCCTTTCCTAATAAAATGAGGAAATTGCATCGCATTGTCTGAG
TAGGTGTCATTCTATTCTGGGGGGTGGGGTGGGGCAGGACAGCAAGGGGGAGGATTGGGAAGACA
ATAGCAGGCATGCTGGGGATGCGGTGGGCTCTATGGCTTCTGAGGCGGAAAGAACCAGATCCTCT
CTTAAGGTAGCATCGAGATTTAAATTAGGGATAACAGGGTAATGGCGCGGGCCGCAGGAACCCCT
AGTGATGGAGTTGGCCACTCCCTCTCTGCGCGCTCGCTCGCTCACTGAGGCCGGGCGACCAAAGG
TCGCCCGACGCCCGGGCTTTGCCCGGGCGGCCTCAGTGAGCGAGCGAGCGCGCAGCTGCCTGCAG
GGGCGCCTGATGCGGTATTTTCTCCTTACGCATCTGTGCGGTATTTCACACCGCATACGTCAAAG
CAACCATAGTACGCGCCCTGTAGCGGCGCATTAAGCGCGGCGGGTGTGGTGGTTACGCGCAGCGT
GACCGCTACACTTGCCAGCGCCCTAGCGCCCGCTCCTTTCGCTTTCTTCCCTTCCTTTCTCGCCA
CGTTCGCCGGCTTTCCCCGTCAAGCTCTAAATCGGGGGCTCCCTTTAGGGTTCCGATTTAGTGCT
TTACGGCACCTCGACCCCAAAAAACTTGATTTGGGTGATGGTTCACGTAGTGGGCCATCGCCCTG
ATAGACGGTTTTTCGCCCTTTGACGTTGGAGTCCACGTTCTTTAATAGTGGACTCTTGTTCCAAA
CTGGAACAACACTCAACCCTATCTCGGGCTATTCTTTTGATTTATAAGGGATTTTGCCGATTTCG
GCCTATTGGTTAAAAAATGAGCTGATTTAACAAAAATTTAACGCGAATTTTAACAAAATATTAAC
GTTTACAATTTTATGGTGCACTCTCAGTACAATCTGCTCTGATGCCGCATAGTTAAGCCAGCCCC
GACACCCGCCAACACCCGCTGACGCGCCCTGACGGGCTTGTCTGCTCCCGGCATCCGCTTACAGA
CAAGCTGTGACCGTCTCCGGGAGCTGCATGTGTCAGAGGTTTTCACCGTCATCACCGAAACGCGC
GAGACGAAAGGGCCTCGTGATACGCCTATTTTTATAGGTTAATGTCATGATAATAATGGTTTCTT
AGACGTCAGGTGGCACTTTTCGGGGAAATGTGCGCGGAACCCCTATTTGTTTATTTTTCTAAATA
CATTCAAATATGTATCCGCTCATGAGACAATAACCCTGATAAATGCTTCAATAATATTGAAAAAG
GAAGAGTATGAGTATTCAACATTTCCGTGTCGCCCTTATTCCCTTTTTTGCGGCATTTTGCCTTC
CTGTTTTTGCTCACCCAGAAACGCTGGTGAAAGTAAAAGATGCTGAAGATCAGTTGGGTGCACGA
GTGGGTTACATCGAACTGGATCTCAACAGCGGTAAGATCCTTGAGAGTTTTCGCCCCGAAGAACG
TTTTCCAATGATGAGCACTTTTAAAGTTCTGCTATGTGGCGCGGTATTATCCCGTATTGACGCCG
GGCAAGAGCAACTCGGTCGCCGCATACACTATTCTCAGAATGACTTGGTTGAGTACTCACCAGTC
ACAGAAAAGCATCTTACGGATGGCATGACAGTAAGAGAATTATGCAGTGCTGCCATAACCATGAG
TGATAACACTGCGGCCAACTTACTTCTGACAACGATCGGAGGACCGAAGGAGCTAACCGCTTTTT
TGCACAACATGGGGGATCATGTAACTCGCCTTGATCGTTGGGAACCGGAGCTGAATGAAGCCATA
CCAAACGACGAGCGTGACACCACGATGCCTGTAGCAATGGCAACAACGTTGCGCAAACTATTAAC
TGGCGAACTACTTACTCTAGCTTCCCGGCAACAATTAATAGACTGGATGGAGGCGGATAAAGTTG
CAGGACCACTTCTGCGCTCGGCCCTTCCGGCTGGCTGGTTTATTGCTGATAAATCTGGAGCCGGT
GAGCGTGGGTCTCGCGGTATCATTGCAGCACTGGGGCCAGATGGTAAGCCCTCCCGTATCGTAGT
TATCTACACGACGGGGAGTCAGGCAACTATGGATGAACGAAATAGACAGATCGCTGAGATAGGTG
CCTCACTGATTAAGCATTGGTAACTGTCAGACCAAGTTTACTCATATATACTTTAGATTGATTTA
AAACTTCATTTTTAATTTAAAAGGATCTAGGTGAAGATCCTTTTTGATAATCTCATGACCAAAAT
CCCTTAACGTGAGTTTTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGCA
GATACCAAATACTGTCCTTCTAGTGTAGCCGTAGTTAGGCCACCACTTCAAGAACTCTGTAGCAC
CGCCTACATACCTCGCTCTGCTAATCCTGTTACCAGTGGCTGCTGCCAGTGGCGATAAGTCGTGT
CTTACCGGGTTGGACTCAAGACGATAGTTACCGGATAAGGCGCAGCGGTCGGGCTGAACGGGGGG
TTCGTGCACACAGCCCAGCTTGGAGCGAACGACCTACACCGAACTGAGATACCTACAGCGTGAGC
TATGAGAAAGCGCCACGCTTCCCGAAGGGAGAAAGGCGGACAGGTATCCGGTAAGCGGCAGGGTC
GGAACAGGAGAGCGCACGAGGGAGCTTCCAGGGGGAAACGCCTGGTATCTTTATAGTCCTGTCGG
GTTTCGCCACCTCTGACTTGAGCGTCGATTTTTGTGATGCTCGTCAGGGGGGCGGAGCCTATGGA
AAAACGCCAGCAACGCGGCCTTTTTACGGTTCCTGGCCTTTTGCTGGCCTTTTGCTCACATGT
2.
SEQ ID NO: 2-Wildtype ChAT Sequence
(GenBank: BC130615.1; Refseq: BC130615; Coding sequence
is from 34-1926 base pairs (bps); Sequence length is
1893bps)
ATGGCAGCAAAAACTCCCAGCAGTGAGGAGTCTGGGCTGCCCAAACTGCCCGTGCCCCCGCTGCA
GCAGACCCTGGCCACGTACCTGCAGTGCATGCGACACTTGGTGTCTGAGGAGCAGTTCAGGAAGA
GCCAGGCCATTGTGCAGCAGTTTGGGGCCCCTGGTGGCCTCGGCGAGACCCTGCAGCAGAAACTC
CTGGAGCGGCAGGAGAAGACAGCCAACTGGGTGTCTGAGTACTGGCTGAATGACATGTATCTCAA
CAACCGCCTGGCCCTGCCTGTCAACTCCAGCCCTGCCGTGATCTTTGCTCGGCAGCACTTCCCTG
GCACCGATGACCAGCTGAGGTTTGCAGCCAGCCTCATCTCTGGTGTACTCAGCTACAAGGCCCTG
CTGGACAGCCACTCCATTCCCACTGACTGTGCCAAAGGCCAGCTGTCAGGGCAGCCCCTTTGCAT
GAAGCAATACTATGGGCTCTTCTCCTCCTACCGGCTCCCCGGCCATACCCAGGACACGCTGGTGG
CTCAGAACAGCAGCATCATGCCGGAGCCTGAGCACGTCATCGTAGCCTGCTGCAATCAGTTCTTT
GTCTTGGATGTTGTCATTAATTTCCGCCGTCTCAGTGAGGGGGATCTGTTCACTCAGTTGAGAAA
GATAGTCAAAATGGCTTCCAACGAGGACGAGCGTTTGCCTCCAATTGGCCTGCTGACGTCTGACG
GGAGGAGCGAGTGGGCCGAGGCCAGGACGGTCCTCGTGAAAGACTCCACCAACCGGGACTCGCTG
GACATGATTGAGCGCTGCATCTGCCTTGTATGCCTGGACGCGCCAGGAGGCGTGGAGCTCAGCGA
CACCCACAGGGCACTCCAGCTCCTTCACGGCGGAGGCTACAGCAAGAACGGGGCCAATCGCTGGT
ACGACAAGTCCCTGCAGTTTGTGGTGGGCCGAGACGGCACCTGCGGTGTGGTGTGCGAACACTCC
CCATTCGATGGCATCGTCCTGGTGCAGTGCACTGAGCATCTGCTCAAGCACATGACGCAGAGCAG
CAGGAAGCTGATCCGAGCAGACTCCGTCAGCGAGCTCCCCGCCCCCCGGAGGCTGCGGTGGAAAT
GCTCCCCGGAAATTCAAGGCCACTTAGCCTCCTCGGCAGAAAAACTTCAACGAATAGTAAAGAAC
CTTGACTTCATTGTCTATAAGTTTGACAACTATGGGAAAACATTCATTAAGAAGCAGAAATGCAG
CCCTGATGCCTTCATCCAGGTGGCCCTCCAGCTGGCCTTCTACAGGCTCCACCGAAGACTGGTGC
CCACCTACGAGAGCGCGTCCATCCGCCGATTCCAGGAGGGACGCGTGGACAACATCAGATCGGCC
ACTCCAGAGGCACTGGCTTTTGTGAGAGCCGTGACTGACCACAAGGCTGCTGTGCCAGCTTCTGA
GAAGCTTCTGCTCCTGAAGGATGCCATCCGTGCCCAGACTGCATACACAGTCATGGCCATAACAG
GGATGGCCATTGACAACCACCTGCTGGCACTGCGGGAGCTGGCCCGGGCCATGTGCAAGGAGCTG
CCCGAGATGTTCATGGATGAAACCTACCTGATGAGCAACCGGTTTGTCCTCTCCACTAGCCAGGT
GCCCACAACCACGGAGATGTTCTGCTGCTATGGTCCTGTGGTCCCAAATGGGTATGGTGCCTGCT
ACAACCCCCAGCCAGAGACCATCCTTTTCTGCATCTCTAGCTTTCACAGCTGCAAAGAGACTTCT
TCTAGCAAGTTTGCAAAAGCTGTGGAAGAAAGCCTCATTGACATGAGAGACCTCTGCAGTCTGCT
GCCGCCTACTGAGAGCAAGCCATTGGCAACAAAGGAAAAAGCCACGAGGCCCAGCCAGGGACACC
AACCTTGA
3. SEQ ID NO: 3-
Human choline acetyltransferase (hChAT) Forward Primer
TGTCATTAATTTCCGCCGTCTC
4. SEQ ID NO: 4-
Human choline acetyltransferase (hChAT) Reverse Primer
GTCCTCGTTGGAAGCCATTT
5. SEQ ID NO: 5-
Glyceraldehyde-3-phosphate dehydrogenase (GAPDH) Forward Primer
CACTCACGGCAAATTCAACGGCA
6. SEQ ID NO: 6-
Glyceraldehyde-3-phosphate dehydrogenase (GAPDH) Reverse Primer
ATGACCCTTTTGGCTCCACCCTT

What is claimed is:

1. A method of treating Alzheimer's disease (AD) in a subject in need thereof, the method comprising administering to the subject a pharmaceutically effective amount of a composition comprising a replication-deficient recombinant adeno-associated virus (AAV) vector and a pharmaceutically acceptable carrier, wherein the AAV vector comprises a polynucleotide encoding a choline acetyltransferase (ChAT) polypeptide.

2. The method of claim 1, wherein the AAV vector comprises at least 70% sequence identity to SEQ ID NO: 1.

3. The method of claim 1, wherein the AAV vector comprises SEQ ID NO: 1.

4. The method of claim 1, wherein the AAV vector further comprises a cytomegalovirus (CMV) promoter.

5. The method of claim 1, wherein the AAV vector comprises an AAV9 vector.

6. The method of claim 1, wherein the polynucleotide encoding a choline acetyltransferase (ChAT) polypeptide comprises at least 70% sequence identity to SEQ ID NO: 2.

7. The method of claim 1, wherein the composition decreases or inhibits AD progression relative to a control.

8. The method of claim 1, wherein the method delivers the vector to the subject's central nervous system.

9. The method of claim 1, wherein the composition is administered using an ocular gene delivery.

10. The method of claim 1, wherein the composition increases expression of the ChAT polypeptide relative to a control composition.

11. The method of claim 1, wherein the composition improves mitochondrial dynamics relative to a control composition.

12. The method of claim 1, wherein the composition treats or prevents cell death, inflammation, and cognitive dysfunction in the subject relative to a control composition.

13. A composition comprising a replication-deficient recombinant adeno-associated virus (AAV) vector comprising a polynucleotide and a pharmaceutically acceptable carrier, wherein the polynucleotide encodes a choline acetyltransferase (ChAT) protein.

14. The composition of claim 13, wherein the AAV vector comprises at least 70% sequence identity to SEQ ID NO: 1.

15. The composition of claim 13, wherein the AAV vector comprises SEQ ID NO: 1.

16. The composition of claim 13, wherein the AAV vector further comprises a cytomegalovirus (CMV) promoter.

17. The composition of claim 13, wherein the AAV vector comprises an AAV9 vector.

18. The composition of claim 13, wherein the polynucleotide encoding a choline acetyltransferase (ChAT) polypeptide comprises at least 70% sequence identity to SEQ ID NO: 2.

19. The composition of claim 13, wherein the composition is a ChAT gene therapeutic agent.

20. The composition of claim 13, wherein the composition is an ocular ChAT gene therapeutic agent.


SEQUENCE LISTING (6 sequences)

SEQ ID NO: 1 - DNA, 6108 bp, synthetic construct
  source 1..6108: mol_type = other DNA
  misc_feature 1..6108: note = replication-deficient recombinant adeno-associated virus (AAV) vector with ChAT
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccaattcagtcgataactataacggtcctaaggtagcgatttaaatacgcgctctcttaaggtagccccgggacgcgtcaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagattgattatggcagcaaaaactcccagcagtgaggagtctgggctgcccaaactgcccgtgcccccgctgcagcagaccctggccacgtacctgcagtgcatgcgacacttggtgtctgaggagcagttcaggaagagccaggccattgtgcagcagtttggggcccctggtggcctcggcgagaccctgcagcagaaactcctggagcggcaggagaagacagccaactgggtgtctgagtactggctgaatgacatgtatctcaacaaccgcctggccctgcctgtcaactccagccctgccgtgatctttgctcggcagcacttccctggcaccgatgaccagctgaggtttgcagccagcctcatctctggtgtactcagctacaaggccctgctggacagccactccattcccactgactgtgccaaaggccagctgtcagggcagcccctttgcatgaagcaatactatgggctcttctcctcctaccggctccccggccatacccaggacacgctggtggctcagaacagcagcatcatgccggagcctgagcacgtcatcgtagcctgctgcaatcagttctttgtcttggatgttgtcattaatttccgccgtctcagtgagggggatctgttcactcagttgagaaagatagtcaaaatggcttccaacgaggacgagcgtttgcctccaattggcctgctgacgtctgacgggaggagcgagtgggccgaggccaggacggtcctcgtgaaagactccaccaaccgggactcgctggacatgattgagcgctgcatctgccttgtatgcctggacgcgccaggaggcgtggagctcagcgacacccacagggcactccagctccttcacggcggaggctacagcaagaacggggccaatcgctggtacgacaagtccctgcagtttgtggtgggccgagacggcacctgcggtgtggtgtgcgaacactccccattcgatggcatcgtcctggtgcagtgcactgagcatctgctcaagcacatgacgcagagcagcaggaagctgatccgagcagactccgtcagcgagctccccgccccccggaggctgcggtggaaatgctccccggaaattcaaggccacttagcctcctcggcagaaaaacttcaacgaatagtaaagaaccttgacttcattgtctataagtttgacaactatgggaaaacattcattaagaagcagaaatgcagccctgatgccttcatccaggtggccctccagctggccttctacaggctccaccgaagactggtgcccacctacgagagcgcgtccatccgccgattccaggagggacgcgtggacaacatcagatcggccactccagaggcactggcttttgtgagagccgtgactgaccacaaggctgctgtgccagcttctgagaagcttctgctcctgaaggatgccatccgtgcccagactgcatacacagtcatggccataacagggatggccattgacaaccacctgctggcactgcgggagctggcccgggccatgtgcaaggagctgcccgagatgttcatggatgaaacctacctgatgagcaaccggtttgtcctctccactagccaggtgcccacaaccacggagatgttctgctgctatggtcctgtggtcccaaatgggtatggtgcctgctacaacccccagccagagaccatccttttctgcatctctagctttcacagctgcaaagagacttcttctagcaagtttgcaaaagctgtggaagaaagcctcattgacatgagagacctctgcagtctgctgccgcctactgagagcaagccattggcaacaaaggaaaaagccacgaggcccagccagggacaccaaccttgaaatcccatccagcacagtggcggccgctcgagtctagagggcccttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagatcctctcttaaggtagcatcgagatttaaattagggataacagggtaatggcgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 2 - DNA, 1893 bp, Homo sapiens
  source 1..1893: mol_type = genomic DNA
  misc_feature 1..1893: note = Wildtype ChAT Sequence (GenBank: BC130615.1; Refseq: BC130615; Coding sequence is from 34-1926 base pairs (bps); Sequence length is 1893bps)
atggcagcaaaaactcccagcagtgaggagtctgggctgcccaaactgcccgtgcccccgctgcagcagaccctggccacgtacctgcagtgcatgcgacacttggtgtctgaggagcagttcaggaagagccaggccattgtgcagcagtttggggcccctggtggcctcggcgagaccctgcagcagaaactcctggagcggcaggagaagacagccaactgggtgtctgagtactggctgaatgacatgtatctcaacaaccgcctggccctgcctgtcaactccagccctgccgtgatctttgctcggcagcacttccctggcaccgatgaccagctgaggtttgcagccagcctcatctctggtgtactcagctacaaggccctgctggacagccactccattcccactgactgtgccaaaggccagctgtcagggcagcccctttgcatgaagcaatactatgggctcttctcctcctaccggctccccggccatacccaggacacgctggtggctcagaacagcagcatcatgccggagcctgagcacgtcatcgtagcctgctgcaatcagttctttgtcttggatgttgtcattaatttccgccgtctcagtgagggggatctgttcactcagttgagaaagatagtcaaaatggcttccaacgaggacgagcgtttgcctccaattggcctgctgacgtctgacgggaggagcgagtgggccgaggccaggacggtcctcgtgaaagactccaccaaccgggactcgctggacatgattgagcgctgcatctgccttgtatgcctggacgcgccaggaggcgtggagctcagcgacacccacagggcactccagctccttcacggcggaggctacagcaagaacggggccaatcgctggtacgacaagtccctgcagtttgtggtgggccgagacggcacctgcggtgtggtgtgcgaacactccccattcgatggcatcgtcctggtgcagtgcactgagcatctgctcaagcacatgacgcagagcagcaggaagctgatccgagcagactccgtcagcgagctccccgccccccggaggctgcggtggaaatgctccccggaaattcaaggccacttagcctcctcggcagaaaaacttcaacgaatagtaaagaaccttgacttcattgtctataagtttgacaactatgggaaaacattcattaagaagcagaaatgcagccctgatgccttcatccaggtggccctccagctggccttctacaggctccaccgaagactggtgcccacctacgagagcgcgtccatccgccgattccaggagggacgcgtggacaacatcagatcggccactccagaggcactggcttttgtgagagccgtgactgaccacaaggctgctgtgccagcttctgagaagcttctgctcctgaaggatgccatccgtgcccagactgcatacacagtcatggccataacagggatggccattgacaaccacctgctggcactgcgggagctggcccgggccatgtgcaaggagctgcccgagatgttcatggatgaaacctacctgatgagcaaccggtttgtcctctccactagccaggtgcccacaaccacggagatgttctgctgctatggtcctgtggtcccaaatgggtatggtgcctgctacaacccccagccagagaccatccttttctgcatctctagctttcacagctgcaaagagacttcttctagcaagtttgcaaaagctgtggaagaaagcctcattgacatgagagacctctgcagtctgctgccgcctactgagagcaagccattggcaacaaaggaaaaagccacgaggcccagccagggacaccaaccttga

SEQ ID NO: 3 - DNA, 22 bp, synthetic construct
  source 1..22: mol_type = other DNA
  misc_feature 1..22: note = Human choline acetyltrasferase (hChAT) Forward Primer
tgtcattaatttccgccgtctc

SEQ ID NO: 4 - DNA, 20 bp, synthetic construct
  source 1..20: mol_type = other DNA
  misc_feature 1..20: note = Human choline acetyltransferase (hChAT) Reverse Primer
gtcctcgttggaagccattt

SEQ ID NO: 5 - DNA, 23 bp, synthetic construct
  source 1..23: mol_type = other DNA
  misc_feature 1..23: note = Glyceraldehyde-3-phosphate dehydrogenase (GAPDH) Forward Primer
cactcacggcaaattcaacggca

SEQ ID NO: 6 - DNA, 23 bp, synthetic construct
  source 1..23: mol_type = other DNA
  misc_feature 1..23: note = Glyceraldehyde-3-phosphate dehydrogenase (GAPDH) Reverse Primer
atgacccttttggctccaccctt